(19) United States
(12) Patent Application Publication
(10) Pub. No.: US 2016/0053227 A1
(43) Pub. Date: Feb. 25, 2016

(54) AGENT FOR INDUCING DIFFERENTIATION OF WHITE ADIPOCYTES INTO BROWN-LIKE ADIPOCYTES

(71) Applicant: UHA MIKAKUTO CO., LTD., Yamatokooriyama-shi (JP)

(72) Inventors: Yasuhiro SHINKA, Yamatokooriyama-shi (JP); Akinobu KISHI, Yamatokooriyama-shi (JP); Taiji MATSUKAWA, Yamatokooriyama-shi (JP); Takeki MATSUI, Yamatokooriyama-shi (JP); Yasumasa YAMADA, Yamatokooriyama-shi (JP); Ichiro YAMADA, Yamatokooriyama-shi (JP)

(73) Assignee: UHA Mikakuto Co., Ltd., Yamatokooriyama-shi (JP)

(21) Appl. No.: 14/781,150

(22) PCT Filed: Feb. 27, 2014
(86) PCT No.: PCT/JP2014/054947

§ 371 Date: Sep. 29, 2015

(30) Foreign Application Priority Data
Mar. 29, 2013 (JP) 2013-071836
Jul. 30, 2013 (JP) 2013-157659
Dec. 25, 2013 (JP) 2013-267473

(51) Int. Cl. C12N 5/077; A61K 31/05; C07C 39/23

(52) U.S. Cl. CPC C12N 5/0653; C07C 39/23; A61K 31/05; C12N 2501/999

(57) ABSTRACT

This invention relates to an agent for inducing differentiation of white adipocytes into brown-like adipocytes comprising a compound which is a cyclization reaction product of a hydroxystilbene and which is represented by Formula (1): (wherein each of R₁ to R₈ is a hydrogen atom, a hydroxyl group, a saturated or unsaturated, straight or branched alkoxy group having 1 to 10 carbon atoms, or a saturated or unsaturated, straight or branched alkyl group having 1 to 10 carbon atoms, and R₁ to R₈ may be same or different), or a pharmaceutically acceptable salt thereof.

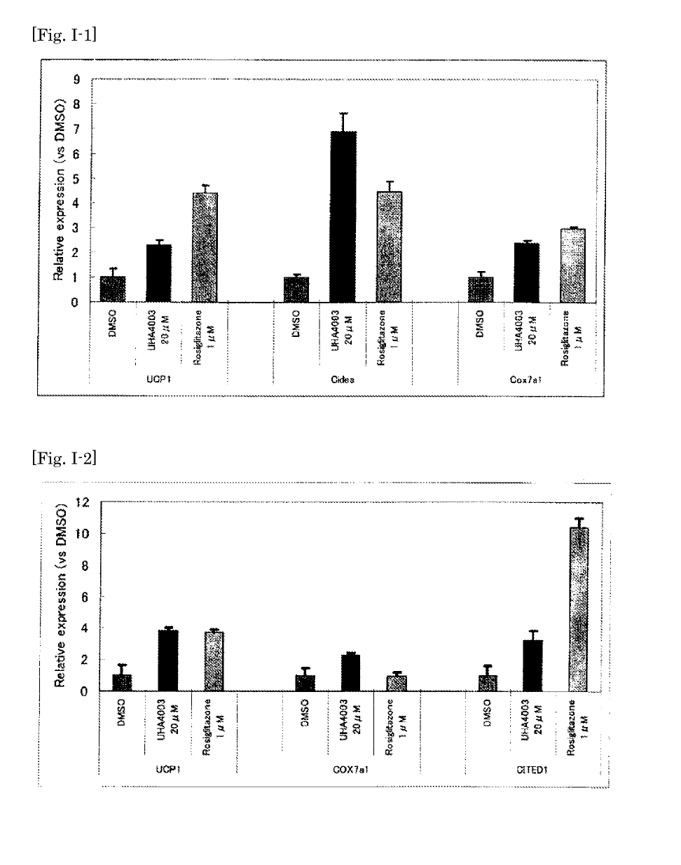

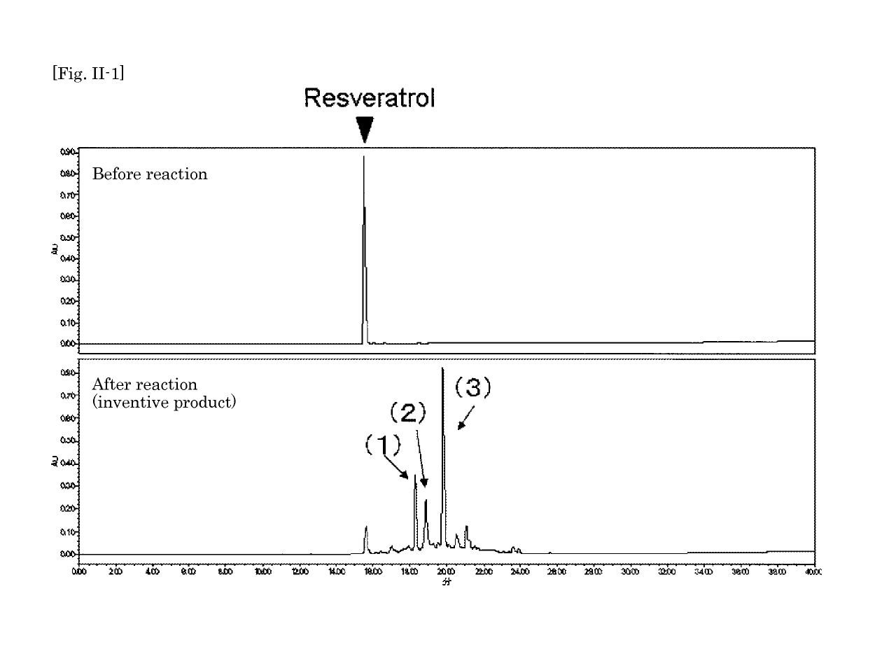

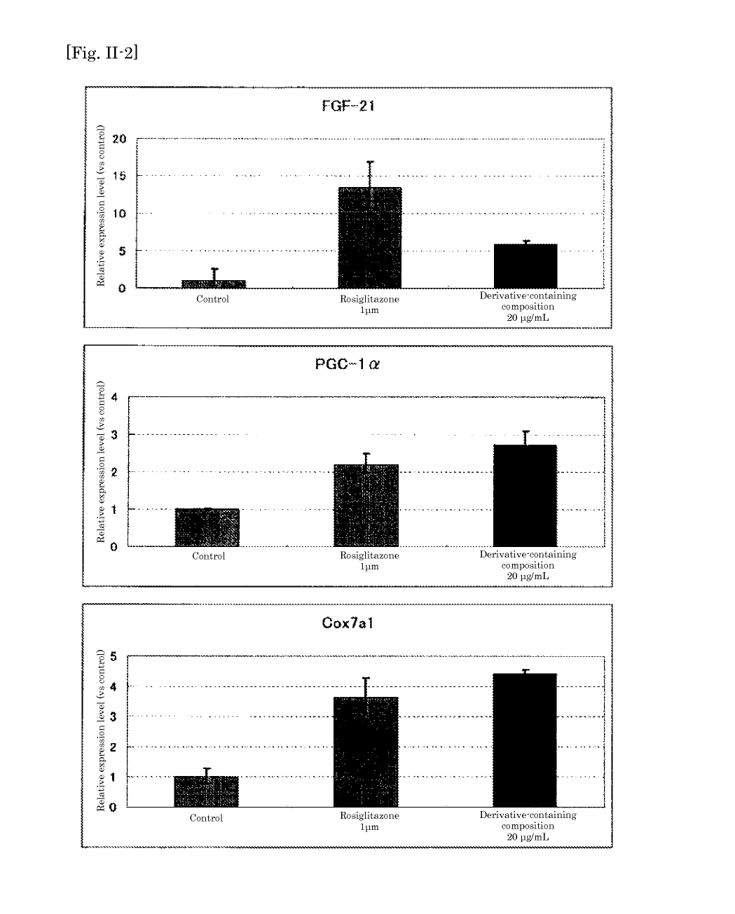

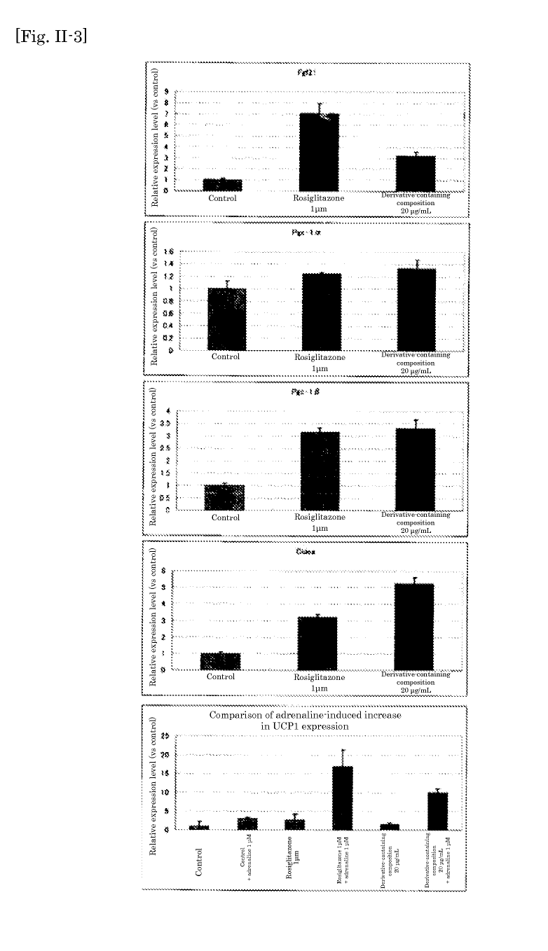

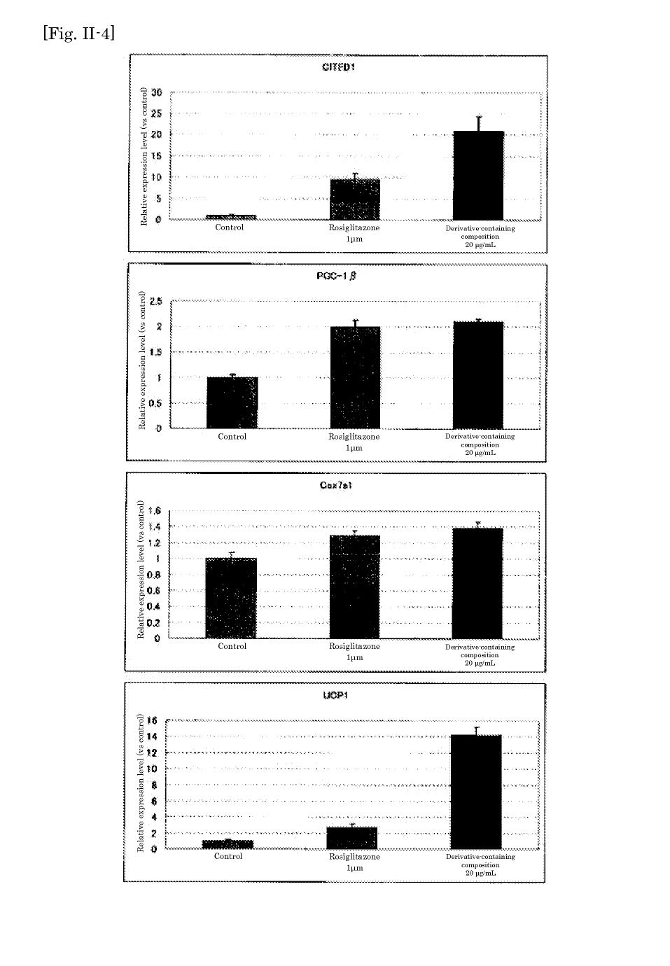

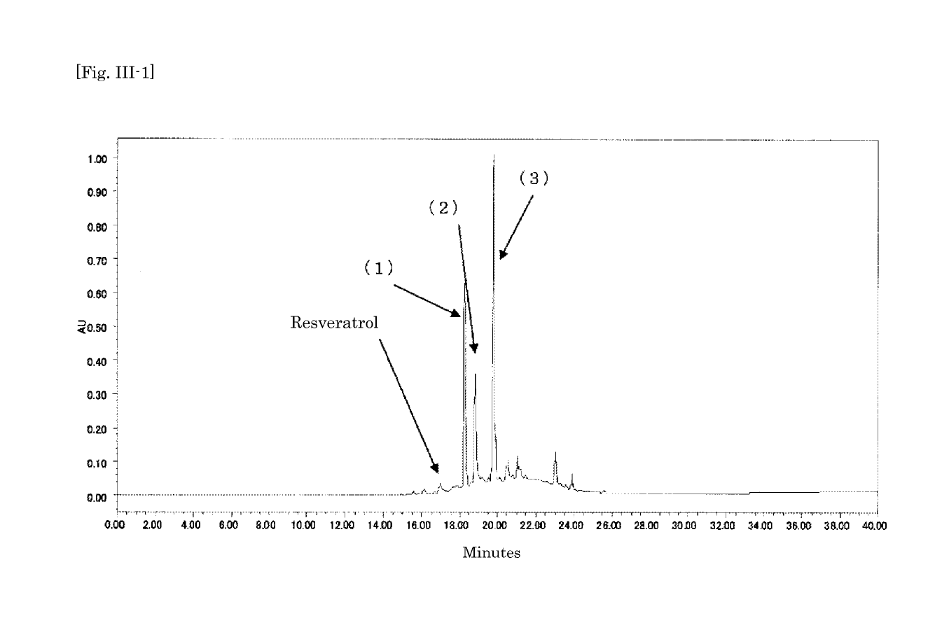

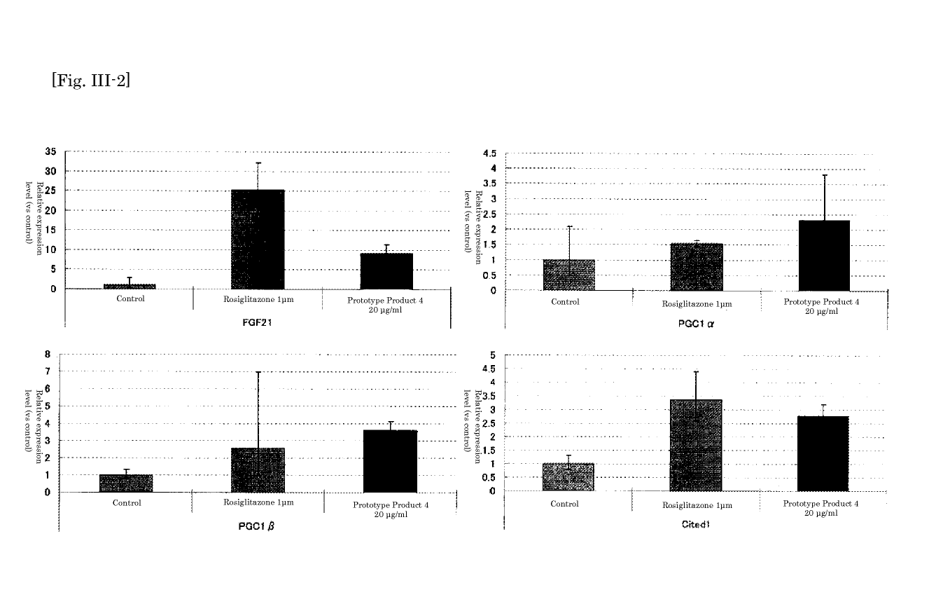

### TECHNICAL FIELD

The present invention relates to an agent for inducing differentiation of white adipocytes into brown-like adipocytes containing a cyclization reaction product of a hydroxystilbene obtained by an extremely simple method which is also a synthetic method applicable even to a food, as well as foods, pharmaceutical agents, and quasi drugs containing the agent for inducing the differentiation of white adipocytes into brown-like adipocytes.

### BACKGROUND ART

Technologies for derivatizing a compound are employed widely in pharmaceutical development and the like for the purpose of enhancing the function of the compound. For example, Tamiflu (trade mark) employed widely as an anti-influenza agent is obtained by derivatizing a shikimic acid contained in a star anise which is a galenical. As another example, pioglitazone, which is an agent for improving insulin resistance, is obtained in a study of derivative synthesis using clofibrate as a lead compound.

While derivatization technologies employed in pharmaceuticals are acceptable when being used in pharmaceuticals even with their complicated synthetic methods and high production costs, they mostly fail to be capable of being used as a highly functional food material or an additive because of their costs and safety rule concerns.

On the other hand, advanced countries now having aging societies are suffering from increased medical bills, and development in preventive medicine for preventing diseases in advance is desired. Unlike to conventional medicines involving symptomatic treatments using pharmaceuticals or particular medical cares, the preventive medicine aims at ensuring precautions for avoiding any disease by ingesting healthy foods in daily life. In such a circumstance, the preventive medicine has a close relationship with foods.

Adipocytes constituting an adipose tissue of a mammal can be grouped into two classes, namely, white adipocytes and brown adipocytes. The white adipocytes mainly play a role for allowing energies once ingested to be accumulated as triglycerides, while the brown adipocytes play a role for converting the accumulated energies into heat. The brown adipocytes are originated in myoblasts and contain a large amount of mitochondria inside of their cells thereby exhibiting a brow color, and are the adipocytes which utilize the activities of uncoupling protein 1 (UCP1) expressed in mitochondria to produce a heat, thereby playing a role to convert the accumulated energies into a heat (Non-patent Document 1).

A brown adipose tissue constituted from the brown adipocytes is observed frequently in infancy and its tissue quantity is reduced along with the growth. It is considered conventionally that the brown adipocytes are not expressed from the white adipocytes since the brown adipocytes differ from the white adipocytes in the originating stem cell. Recently, however, it became evident that the white adipose tissue contains “brown-like adipocyte” which exhibits a response similar to that of the brown adipocytes (Non-patent Document 2). The brown-like adipocytes contain the mitochondria in an amount greater substantially than that in the white adipocytes although they are originated from the white adipocytes, and thus are the adipocytes which can convert the accumulated lipid energy efficiently into a heat energy. This report indicates that it is possible to increase the brown-like adipose tissue even in human adults, and the induction of the differentiation of the white adipocytes into the brown-like adipocytes is focused on as a novel and radical therapeutic method against obesity or diabetes.

Nevertheless, it is difficult to induce the white adipocytes into the brown-like adipocytes, and the methods which have been verified are long term cold stimulation, adrenaline stimulation, addition of peroxisome proliferator-activated receptor (PPAR_{Y}) agonist (Non-patent Document 3) and the like, all of which involve the limitation of the differentiation inducing effect only to the subcutaneous adipocytes. While Patent Document 1 reported a compound having a UCP1 gene expression amplifying effect on human visceral adipocytes, no browning of the white adipocytes was observed, and an extremely high effective concentration poses a difficulty in achieving an in vitro effective concentration, because of which the browning of the white adipocytes has not been achieved yet.

On the other hand, currently there are 22 known human fibroblast growth factors (FGFs) based on the homology in the amino acid sequences (Non-patent Document 4). FGF-21 was reported to have a possibility of being involved in promotion of lipolysis and suppression of fat accumulation, improvement in hypercholesterolemia, diabetes (hyperglycemia, insulin resistance), and obesity (Non-patent Document 5). For example, Patent Document 1 describes a compound which promotes FGF-21 production and is effective in reducing the visceral fat level, while Patent Document 2 describes that type 2 diabetes treating agent consisting of FGF-21 and glucagon-like peptide-1 receptor agonist, anti-diabetic agent, dipeptidyl peptidase-4 inhibitor is useful. Patent Document 3 also describes that it is useful in treating metabolic diseases to use FGF-21 derivatives for prolonging the half life of FGF-21. In such a circumstance, it was reported recently that a cold stimulation promotes the elevation of the blood FGF-21 level thereby promoting the browning of the white adipocytes, and FGF-21 is focused on as one of the factors for inducing browning of the white adipocytes (Non-patent Document 6). Nevertheless, the reports targeted to FGF-21 are limited to those aiming at improvement of metabolic failures, and are not proposed typically to be used as a means for browning the white adipocytes.

On the other hand, useful physiological effect of a red wine, which is called “French paradox”, is considered to be attributable partly to various bioactive functions including antioxidative ability of resveratrol. Resveratrol is one of hydroxystilbenes contained in grape pericarp or peanut red skin at high levels, and known as a plant-derived compound having various activities including a sirtuin-mediated calorie limiting effect, anti-mycotic, anti-bacterial, anti-inflammatory activities, and the like (Non-patent Document 7). Furthermore, resveratrol was reported to have suppressive effects on sirtuin expression promotion-mediated differentiation into adipocytes and also on fat accumulation (Non-patent Document 8). Nevertheless, it is still unclear that the sirtuin-mediated calorie limiting effect possessed by resveratrol is effective also in humans in such a circumstance that the results of the experiment in rhesus monkeys indicated that it is doubtful that the practice of the calorie limitation itself is useful actually in prolonging the life (Non-patent Document 9). While resveratrol was reported also to be involved in the gene expression promoting effect for an uncoupling protein UCP2 expressed specifically on the white adipocytes (Patent Document 4), there are no mentions of effects which differentiate the white adipocytes into the brown-like adipocytes so far with regard to resveratrol.

As discussed above, it has been desired to develop radical therapeutic or prophylactic agents as well as functional foods containing the same to prevent or treat the metabolic syndrome by changing the nature of an adipocyte itself thereby change the metabolism rather than by suppressing appetite or by suppressing fat absorption, but a substance which exhibits a sufficient effect on all white adipocytes has not been found yet and is still desired to be developed as soon as possible.

Currently, a large number of compounds are obtained by using various methods such as fermentation, extraction, organic synthesis, and the like, and utilized in a wide range of the fields including food products and pharmaceuticals, however, those which have both of the usefulness and the practicability are limited. In addition, novel compounds, which have not been reported yet, are considered to be further difficult in obtaining both of the usefulness and the practicability because of the cost of raw materials for synthesis as well as the technical difficulty in isolation.

In such a circumstance, we found a unique method for synthesizing novel compounds which allows the isolation to be performed easily, which is simple and convenient, and which uses components derived from foods (Patent Document 5), and such compounds are expected to be used in a wider range of applications.

### CITATION LIST

### Patent Literatures

Patent Document 1: JP-A No. 2011-241195
Patent Document 2: National Publication No. 2013-518035
Patent Document 3: National Publication No. 2012-515747
Patent Document 4: JP-A No. 2010-24208
Patent Document 5: International Publication WO2013/061455

### Non-Patent Literatures

Non-patent Document 1: Cell Biosci. 2011 Oct. 28; 1:35
Non-patent Document 2: Cell. 2012 Jul. 20; 150(2):366-376
Non-patent Document 3: Nat Med. 2013 October; 19(10):1252-63
Non-patent Document 4: Genome Biol. 2001; 2(3):REVIEWS 3005
Non-patent Document 5: Am J Clin Nutr. 2010 January; 91(1): 254S?257S
Non-patent Document 6: Genes Dev. 2012 Feb. 1; 26(3):271-81
Non-patent Document 7: Ed. by Kazuo Tsubota, “Basic and applied sciences of resveratrol”, CMC Publishing Co., Ltd., 2012
Non-patent Document 8: Nature. 2004 Jun. 17; 429(6993):771
Non-patent Document 9: Nature. 2012 Sep. 13; 489(7415):318

### SUMMARY OF INVENTION

### Technical Problem

We made an intensive study, in view of the circumstance described above, for searching for compounds exhibiting an effect which induces the differentiation of the white adipocytes into the brown-like adipocytes and also for establishing the methods for producing the same, and we were successful unexpectedly in producing compounds exhibiting an effect which induces the differentiation of the white adipocytes into the brown-like adipocytes and the compositions containing the compounds by as a simple, convenient, and safe method as heat treatment of hydroxystilbenes under alkaline conditions, thereby establishing the present invention.

Accordingly, an object of the present invention is to provide an agent for inducing differentiation of the white adipocytes into the brown-like adipocytes containing a novel compound having an excellent ability of differentiating the white adipocytes into the brown-like adipocytes which has not been found yet in hydroxystilbenes such as resveratrol.

Another object of the invention is to provide a composition of an agent for inducing differentiation of the white adipocytes into the brown-like adipocytes which has an excellent ability of differentiating the white adipocytes into the brown-like adipocytes and which can be produced safely and inexpensively, as well as a method for producing the same.

Another object of the invention is to provide a food, pharmaceutical agent, and quasi drug containing the agent for inducing the differentiation of white adipocytes into brown-like adipocytes or composition of the agent for inducing differentiation of the white adipocytes into the brown-like adipocytes.

### Solution to Problem

Thus, the present invention relates to the followings:

[1] An agent for inducing differentiation of white adipocytes into brown-like adipocytes comprising a compound which is a cyclization reaction product of a hydroxystilbene and which is represented by Formula (1):

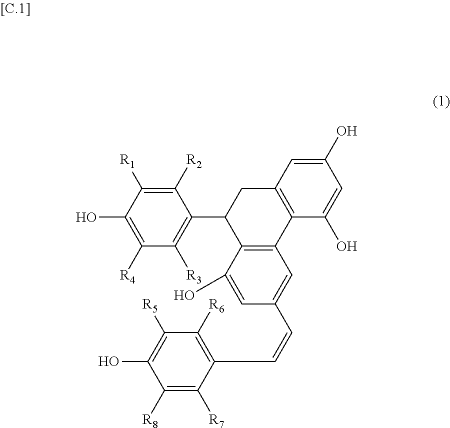

(wherein each of R₁ to R₈ is a hydrogen atom, a hydroxyl group, a saturated or unsaturated, straight or branched alkoxy group having 1 to 10 carbon atoms, or a saturated or unsaturated, straight or branched alkyl group having 1 to 10 carbon atoms, and R₁ to R₈ may be same or different), or a pharmaceutically acceptable salt thereof;
[2] The agent for inducing differentiation of white adipocytes into brown-like adipocytes according to [1] described above wherein the hydroxystilbene is resveratrol;
[3] The agent for inducing differentiation of white adipocytes into brown-like adipocytes according to [1] or [2] described above wherein all of R₁ to R₈ are hydrogen atoms;
[4] A composition of an agent for inducing differentiation of white adipocytes into brown-like adipocytes which is a composition obtained by heating resveratrol under an alkaline condition and which comprises three resveratrol polymerization compounds represented by Formula (2):

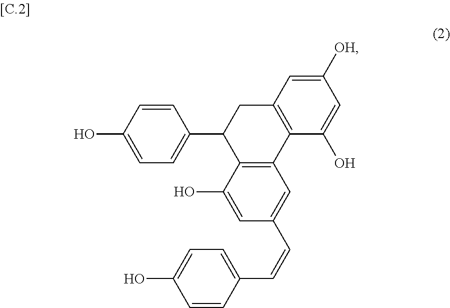 [SMILES: Oc1ccc(/C=C\c2cc(O)c3c(c2)-c2c(O)cc(O)cc2CC3c2ccc(O)cc2)cc1]

### Formula (3):

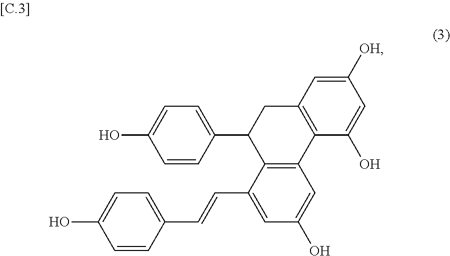 [SMILES: Oc1ccc(/C=C/c2cc(O)cc3c2C(c2ccc(O)cc2)Cc2cc(O)cc(O)c2-3)cc1]

### and Formula (4):

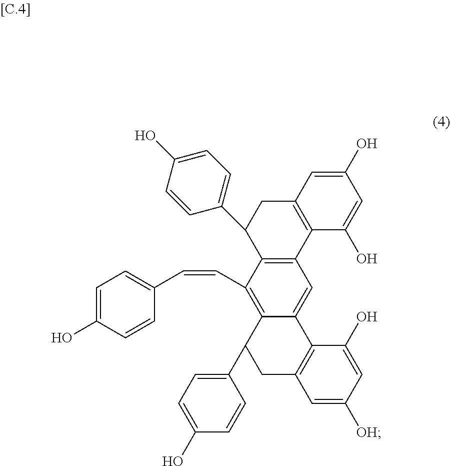 [SMILES: Oc1ccc(/C=C\c2c3c(cc4c2C(c2ccc(O)cc2)Cc2cc(O)cc(O)c2-4)-c2c(O)cc(O)cc2CC3c2ccc(O)cc2)cc1]

[5] The composition of the agent for inducing differentiation of white adipocytes into brown-like adipocytes according to [4] described above wherein the phenol content in 20 mg/mL solid is 100 μg/mL or less;
[6] A food, pharmaceutical agent, and quasi drug comprising the agent for inducing differentiation of white adipocytes into brown-like adipocytes according to any of [1] to [3] described above;
[7] A food, pharmaceutical agent, and quasi drug comprising the composition of the agent for inducing differentiation of white adipocytes into brown-like adipocytes according to [4] or [5] described above;
[8] A method for producing the composition of the agent for inducing differentiation of white adipocytes into brown-like adipocytes according to [4] or [5] described above which comprises a step for removing phenol contained therein by one or more steps selected from the group consisting of Steps (a) to (c):
(a) a step for allowing a solution obtained by heating resveratrol under an alkaline condition to be brought into contact with a solid adsorbent and recovering the adsorbed constituents;
(b) a step for adjusting a solution obtained by heating resveratrol under an alkaline condition to pH7 or below to effect precipitation and recovering the precipitated material; and,
(c) a step for heating a solution obtained by heating resveratrol under an alkaline condition, the composition obtained by the Step (a), or an aqueous solution containing the composition obtained by the Step (b).

### Advantageous Effects of Invention

The compound represented by Formula (1) shown above and a pharmaceutically acceptable salt thereof employed in this invention have an effect which induces differentiation of white adipocytes into brown-like adipocytes which is more excellent than those of its precursors hydroxystilbenes, and are useful as a novel therapeutic and prophylactic substance against metabolic syndrome.

Since the composition of the agent for inducing differentiation of white adipocytes into brown-like adipocytes of this invention (hereinafter referred to also as the composition of this invention) can be produced safely and inexpensively by heating resveratrol under an alkaline condition and has a remarkably excellent effect which induces differentiation of white adipocytes into brown-like adipocytes which is a novel effect which is not possessed by a precursor resveratrol, it is useful in a novel therapeutic and prophylactic treatment against the metabolic syndrome.

Since the composition of this invention has an excellent effect which induces differentiation of white adipocytes into brown-like adipocytes without conducting any isolation and purification steps of the compound represented by Formula (1) shown above and a pharmaceutically acceptable salt thereof, it is possible to provide an agent for inducing differentiation of white adipocytes into brown-like adipocytes with a higher producibility at a lower cost.

Also since the composition of this invention can provide a safer resveratrol heat treatment composition as a result of an efficient reduction in phenol which tends to be generated upon heating resveratrol under an alkaline condition.

Also by incorporating an agent for inducing differentiation of white adipocytes into brown-like adipocytes or a composition according to the invention into a food, pharmaceutical agent, and quasi drug, it is possible to provide a novel food, pharmaceutical agent, and quasi drug for preventing or improving the metabolic syndrome.

### BRIEF DESCRIPTION OF THE DRAWINGS

FIG. I-1 is a graph showing the relative gene expression levels of UCP1 gene, cell death-inducing DFFA-like effector a (Cidea) which is a brown-like adipocyte marker gene, and cytochrome c oxidase polypeptide 7A1 (Cox7a1) which is a mitochondria marker gene in adipocytes after inducing differentiation in Example I-2.

FIG. I-2 is a graph showing the relative gene expression levels of UCP1 gene, cytochrome c oxidase polypeptide 7A1 (Cox7a1) which is a mitochondria marker gene, and CBP/p300-interacting transactivator (CITED1) which is a brown-like adipocyte marker gene in adipocytes after inducing differentiation of normal subcutaneous fat in Example I-3.

FIG. II-1 shows chromatograms of the resveratrol reaction solution in Example II-1.

FIG. II-2 is a graph showing the relative gene expression levels of FGF-21, peroxisome proliferator-activated receptor_{Y} coactivator 1α (PGC-1α) which is a transcription coactivator, and cytochrome c oxidase polypeptide 7A1 (Cox7a1) which is a mitochondria marker gene in adipocytes immediately after inducing differentiation in Example II-2.

FIG. II-3 is a graph showing the relative gene expression levels of FGF-21, PGC-1α, Cidea, peroxisome proliferator-activated receptor_{Y} coactivator 16 (PGC-1β) which is a transcription coactivator, and UCP1 in adipocytes after maturation in Example II-3.

FIG. II-4 is a graph showing the relative gene expression levels of PGC-1β, UCP1, COX7a1, and CITED1 in adipocytes after inducing differentiation of normal subcutaneous fat in Example II-4.

FIG. III-1 shows a chromatogram of the resveratrol reaction solution in Example III-1.

FIG. III-2 is a graph showing the relative gene expression levels of FGF-21, PGC-1α, PGC-1β, and CITED1 in adipocytes immediately after inducing differentiation in Example III-2.

### DESCRIPTION OF EMBODIMENTS

The invention is described in detail below.

The cyclization reaction product of a hydroxystilbene employed in this invention is a compound represented by Formula (1):

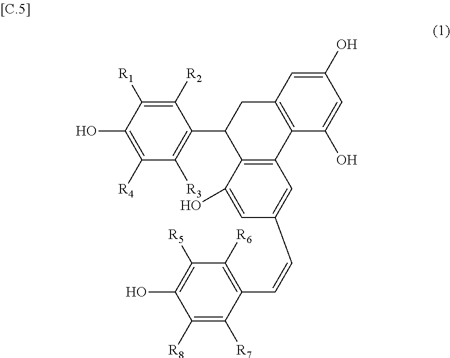

(wherein each of R₁ to R₈ is a hydrogen atom, a hydroxyl group, a saturated or unsaturated, straight or branched alkoxy group having 1 to 10 carbon atoms, or a saturated or unsaturated, straight or branched alkyl group having 1 to 10 carbon atoms, and R₁ to R₈ may be same or different), or a pharmaceutically acceptable salt thereof.

In Formula (1) shown above, the saturated or unsaturated, straight or branched alkoxy group having 1 to 10 carbon atoms represented by R₁ to R₈ is not limited particularly, and is preferably a straight or branched alkoxy group having 1 to 4 carbon atoms. Those exemplified typically are methoxy group, ethoxy group, n-propoxy group, isopropoxy group, n-butoxy group, isobutoxy group, s-butoxy group, t-butoxy group, and the like.

The saturated or unsaturated, straight or branched alkyl group having 1 to 10 carbon atoms represented by R₁ to R₈ is not limited particularly, and is preferably a straight or branched alkyl group having 1 to 5 carbon atoms. Those exemplified typically are methyl group, ethyl group, n-propyl group, isopropyl group, n-butyl group, isobutyl group, s-butyl group, t-butyl group, n-pentyl group, isopentyl group, t-pentyl group, neopentyl group, and the like.

In those listed above, it is preferable that one or more of the R₁ to R₈ is a hydrogen atom, and it is more preferable that all of R₁ to R₈ are hydrogen atoms.

In the compound represented by Formula (1) shown above, the carbon-carbon double bond may be in a trans or cis form. The compound represented by Formula (1) shown above may be a mixture of the cis form and the trans form.

Examples of the pharmaceutically acceptable salt of the compound represented by Formula (1) shown above may for example be alkaline metal salts such as lithium salt, sodium salt, and potassium salt; alkaline earth metal salts such as magnesium salt, calcium salt, and barium salt; aluminum salt; metal hydroxide salts such as aluminum hydroxide salt; amine salts such as alkyl amine salt, dialkyl amine salt, trialkyl amine salt, alkylene diamine salt, cycloalkyl amine salt, aryl amine salt, aralkyl amine salt, and heterocyclic amine salt; amino acid salts such as α-amino acid salt and ω-amino acid salt; peptide salt or a primary, secondary, tertiary, or quaternary amine salt derived therefrom, and the like. These pharmaceutically acceptable salts can be used alone or as a mixture of two or more thereof.

The compound represented by Formula (1) shown above and a pharmaceutically acceptable salt thereof (hereinafter referred to also as the compound represented by Formula (1) shown above) have an effect which induces differentiation of the white adipocytes into the brown-like adipocytes. Such an adipocyte differentiation inducing effect can typically be measured by the methods found in Examples I-2 and I-3 described below.

The compounds represented by Formula (1) shown above can be obtained by subjecting hydroxystilbenes as starting compounds to a heat treatment under an alkaline condition.

The hydroxystilbenes are the hydroxystilbene derivatives represented by Formula (5):

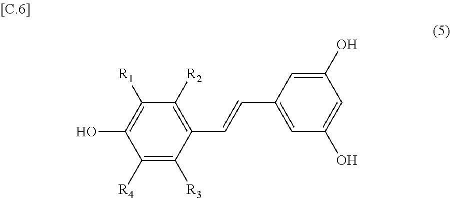

(wherein each of R₁ to R₄ is a hydrogen atom, a hydroxyl group, a saturated or unsaturated, straight or branched alkoxy group having 1 to 10 carbon atoms or a saturated or unsaturated, straight or branched alkyl group having 1 to 10 carbon atoms, and R₁ to R₄ may be same or different), and pharmaceutically acceptable salts thereof.

In Formula (5) shown above, the saturated or unsaturated, straight or branched alkoxy group having 1 to 10 carbon atoms represented by R₁ to R₄ is not limited particularly, and is preferably a straight or branched alkoxy group having 1 to 4 carbon atoms. Those exemplified typically are methoxy group, ethoxy group, n-propoxy group, isopropoxy group, n-butoxy group, isobutoxy group, s-butoxy group, t-butoxy group, and the like.

The saturated or unsaturated, straight or branched alkyl group having 1 to 10 carbon atoms represented by R₁ to R₄ is not limited particularly, and is preferably a straight or branched alkyl group having 1 to 5 carbon atoms. Those exemplified typically are methyl group, ethyl group, n-propyl group, isopropyl group, n-butyl group, isobutyl group, s-butyl group, t-butyl group, n-pentyl group, isopentyl group, t-pentyl group, neopentyl group, and the like. In those listed above, it is preferable that one or more of the R₁ to R₄ is a hydrogen atom, and resveratrol is more preferred in which all of R₁ to R₄ are hydrogen atoms.

Examples of the pharmaceutically acceptable salt of the compound represented by Formula (5) shown above may for example be alkaline metal salts such as lithium salt, sodium salt, and potassium salt; alkaline earth metal salts such as magnesium salt, calcium salt, and barium salt; aluminum salt; metal hydroxide salts such as aluminum hydroxide salt; amine salts such as alkyl amine salt, dialkyl amine salt, trialkyl amine salt, alkylene diamine salt, cycloalkyl amine salt, aryl amine salt, aralkyl amine salt, and heterocyclic amine salt; amino acid salts such as α-amino acid salt and co-amino acid salt; peptide salt or a primary, secondary, tertiary, or quaternary amine salt derived therefrom, and the like. These pharmaceutically acceptable salts can be used alone or as a mixture of two or more thereof.

The hydroxystilbenes employed as the starting compounds may be naturally-occurring ones or may be highly pure chemical products obtained by chemical synthesis. The naturally-occurring starting compounds may not be those purified completely and may be mixtures containing other compounds.

Nevertheless, for the purpose of obtaining higher production efficiency or % recovery of the compounds represented by Formula (1) shown above, a starting material containing the hydroxystilbenes in an amount in total of 5% by weight or more as the hydroxystilbenes is preferred. Such a starting material may be an extract from a grape pericarp, wine, concentrated wine powder, melinjo, lingonberry, peanut red skin, Japanese knotweed, and the like as well as a freeze-dried material of such an extract. Such an extract may further be purified using a column and the like to obtain a material containing a higher level of hydroxystilbenes such as resveratrol, which is employed preferably.

In the present invention, the hydroxystilbenes are suspended or dissolved in appropriate solvents. Such a solvent may be water or mixtures of water with organic solvents, and dissolution only in organic solvents is also possible. The ratios of water to the organic solvents or the types of the organic solvents are not limited particularly, and complete dissolution may not be required as long as the hydroxystilbenes are thoroughly dispersed. While it is possible to use only water or a mixture such as water and methanol or water and ethanol, it is preferable to use only water in view of safety and cost. When the composition obtained after the heating reaction is employed in a food, pharmaceutical agent, and quasi drug without any sufficient final purification, it is desirable to use ethanol or water, or water-containing ethanol as a solvent in view of safety and legislation.

While the concentration of the hydroxystilbenes in the solution mixture obtained by suspending or dissolving the hydroxystilbenes in the solvents as described above (hereinafter referred to also as hydroxystilbene-containing solutions) is not limited particularly, it is preferable to adjust the hydroxystilbene concentration relative to the each solvent at a level not less than the concentration at which each of the hydroxystilbenes is saturated also in view of the benefit of a smaller amount of the solvents at a higher concentration of the hydroxystilbenes.

Next, the hydroxystilbene-containing solution is adjusted to an alkaline pH. The adjustment may be conducted for example by addition of an basification agent after preparing the hydroxystilbene-containing solution, or it is also possible to adjust the pH of the solvent in advance of the preparation of the hydroxystilbene-containing solution. A pH of 8.0 or higher at the start of the heating reaction of the hydroxystilbene-containing solution is preferable because it allows the reaction described below to proceed efficiently, and a pH exceeding 13.0 allows the reaction to be accompanied with other reaction or target compound degradation, resulting in a reduced yield of the final hydroxystilbene polymerization compounds. Accordingly, a pH at the start of the reaction of 8.0 to 13.0 is desirable.

The basification agent is not limited particularly, and it is preferable, in view of safety, efficiency, and cost, to use sodium hydroxide, potassium hydroxide, sodium hydrogen carbonate, sodium carbonate, and the like. In the case that the change in pH during reaction should be minimized, a buffer solution may be employed, although it is not necessary.

Next, the hydroxystilbene-containing solution adjusted to an alkaline pH is heated. The heat enables a cyclization reaction between the hydroxystilbenes to form the compounds represented by Formula (1) shown above. As used herein, the cyclization reaction is a reaction to form a 6-membered ring as a result of polymerization reaction between the hydroxystilbenes.

Also in the invention, it was confirmed that the cyclization reaction of resveratrol as a hydroxystilbene with each other yields a compound represented by Formula (2):

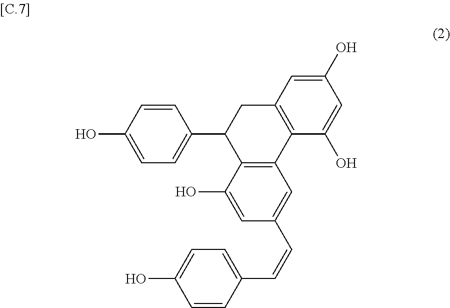 [SMILES: Oc1ccc(/C=C\c2cc(O)c3c(c2)-c2c(O)cc(O)cc2CC3c2ccc(O)cc2)cc1]

and resveratrol polymerization compounds represented by Formula (3):

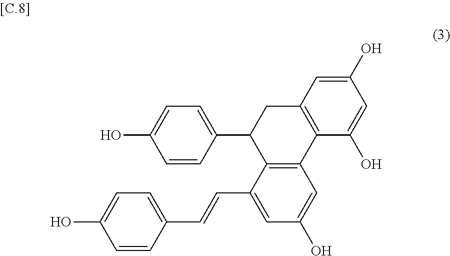 [SMILES: Oc1ccc(/C=C/c2cc(O)cc3c2C(c2ccc(O)cc2)Cc2cc(O)cc(O)c2-3)cc1]

### and by Formula (4):

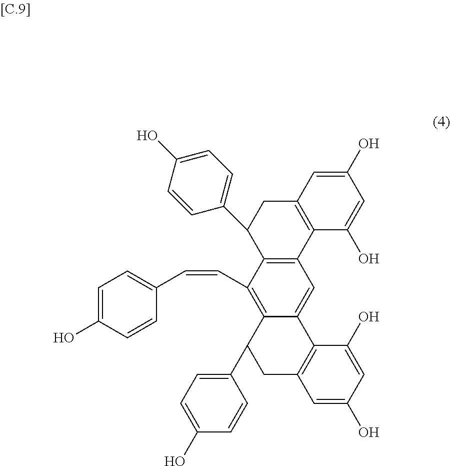 [SMILES: Oc1ccc(/C=C\c2c3c(cc4c2C(c2ccc(O)cc2)Cc2cc(O)cc(O)c2-4)-c2c(O)cc(O)cc2CC3c2ccc(O)cc2)cc1]

additionally.

Also in the resveratrol polymerization compounds represented by Formula (3) or (4) shown above, the carbon-carbon double bond may be in a trans or cis form similarly to the compound represented by Formula (1), and a mixture of the cis form and the trans form is also encompassed.

The resveratrol polymerization compounds represented by Formula (3) or (4) shown above may also be pharmaceutically acceptable salts thereof. The pharmaceutically acceptable salts of the resveratrol polymerization compounds represented by Formula (3) or (4) shown above may, for example, be alkaline metal salts such as lithium salt, sodium salt, and potassium salt; alkaline earth metal salts such as magnesium salt, calcium salt, and barium salt; aluminum salt; metal hydroxide salts such as aluminum hydroxide salt; amine salts such as alkyl amine salt, dialkyl amine salt, trialkyl amine salt, alkylene diamine salt, cycloalkyl amine salt, aryl amine salt, aralkyl amine salt, and heterocyclic amine salt; amino acid salts such as α-amino acid salt and ω-amino acid salt; peptide salt or a primary, secondary, tertiary, or quaternary amine salt derived therefrom, and the like. These pharmaceutically acceptable salts can be used alone or as a mixture of two or more thereof.

Hereinafter the compounds represented by Formula (1) shown above and the resveratrol polymerization compound represented by Formulae (2) to (4) are referred to collectively as the hydroxystilbene cyclization reaction products.

For allowing the cyclization reaction to proceed efficiently, it is preferable that the temperature at which the hydroxystilbene-containing solution is heated is adjusted to 110° C. or higher. Also in view of the boiling points of the solvents employed, heating under pressure is desirable. For example, it is desirable to conduct the heating, for example, by placing the hydroxystilbene-containing solution in an open container which is then heated at a high temperature exceeding the boiling point of the solvent, by placing the hydroxystilbene-containing solution in a tightly closed container which is then heated, by using a retort device or an autoclave to effect heating under pressure and by using an supercritical device or pressure cooker to effect heating under pressure, thereby achieving the temperature of the hydroxystilbene-containing solution of 110° C. or higher at least partly. In view of the recovery efficiency of the hydroxystilbene cyclization reaction products, it is further preferred to keep the temperature of the hydroxystilbene-containing solution uniformly at 120° C. to 180° C. The heating duration is not limited similarly to the heating temperature, and any time period may be employed as long as the intended reaction proceeds efficiently. Especially, the heating duration depends on the heating temperature, and the heating duration appropriate to the heating temperature is desirable. For example, a heating duration of 5 minutes to 270 minutes is desirable for heating at about 130° C. It is possible to adjust the production quantity of the desired compound by adjusting the heating duration. For example, when producing the compound represented by Formula (1), a time period of 5 minutes to 60 minutes is acceptable. On the other hand, when producing the resveratrol polymerization compound represented by any of the Formulae (2) to (4) shown above, a time period of 10 minutes to 270 minutes is acceptable.

Heating may be conducted once or several times repetitively. When the heating is conducted several times, it is preferable that the solvent is newly added.

The termination of the reaction for producing the hydroxystilbene cyclization reaction products by the heating can be verified based on HPLC analysis of the amount of each compound produced.

When the hydroxystilbene cyclization reaction products are produced by steps using only safe starting materials, a mixture containing the hydroxystilbene cyclization reaction products can be used as it is in a food, pharmaceutical agent, and quasi drug as described below. For example, when a naturally-occurring resveratrol is dissolved in a water-containing ethanol solvent, adjusted to an alkaline pH using sodium hydroxide or sodium hydrogen carbonate, and then heated for reaction, then the resultant liquid reaction product can be employed as one of the starting materials for a food, pharmaceutical agent, and quasi drug.

For improving the flavor or achieving a higher function, the reaction product can be concentrated to raise the concentration of the hydroxystilbene cyclization reaction products. If necessary, the reaction product can be purified to obtain a pure product of the compound represented by Formula (1). The concentration or purification can be conducted by known methods. For example, the hydroxystilbene cyclization reaction products can be concentrated by solvent extraction method using chloroform, ethyl acetate, ethanol, methanol, and the like, or by supercritical fluid extraction using carbon dioxide gas. It is also possible to conduct the concentration or the purification by utilizing column chromatography or HPLC. The concentration or purification may use recrystallization, filter process using ultrafiltration membrane, desalting, and the like.

In one method, a synthetic adsorbent is used to adsorb the hydroxystilbene cyclization reaction products, which is thereafter eluted, thereby accomplishing the concentration and the purification easily. The synthetic adsorbent may, for example, be an aromatic synthetic adsorbent such as DIAION (trade mark) HP series and SEPABEADS (trade mark) SP series manufactured by Mitsubishi Chemical Corporation and a styrenic synthetic adsorbent such as Amberlite (trade mark) XAD series manufactured by Organo Corporation.

It is also possible to subject the concentrated or purified material, if necessary, to drying under reduced pressure or freeze drying to remove the solvent, thereby obtaining a powdery solid.

Also when obtaining a composition containing the resveratrol polymerization compounds represented by Formulae (2) to (4) shown above, the solution obtained by heating resveratrol under an alkaline condition as described above (hereinafter referred to also as the reaction solution) tends to generate phenol, which can be removed by subjecting the reaction solution to the treatment described in Steps (a) to (c) shown below.

(a) a step for allowing the reaction solution to be brought into contact with a solid adsorbent and recovering the adsorbed constituents;
(b) a step for adjusting the reaction solution to pH7 or below to effect precipitation and recovering the precipitated material; and,
(c) a step for heating the reaction solution, the composition obtained by the Step (a), or the aqueous solution containing the composition obtained by the Step (b).

Only one of the Steps (a) to (c) may be selected or two or more of them may be combined as long as the phenol concentration of the reaction solution can be reduced. It is also acceptable to repeat an identical step.

The solid adsorbent employed in the Step (a) may, for example, be various synthetic adsorbents or silica gel. The synthetic resin may, for example, be an aromatic synthetic adsorbent such as DIAION (trade mark) HP series and SEPABEADS (trade mark) SP series manufactured by Mitsubishi Chemical Corporation, a styrenic synthetic adsorbent such as Amberlite (trade mark) XAD series manufactured by Organo Corporation, and a soft gel based on dextran or agarose such as Sephadex series and Sepharose series manufactured by GE Healthcare Japan. The silica gel may, for example, be a chemical bond-type porous spherical silica gel whose surface is modified with octadecylsilyl group and the like.

The method for bringing these solid adsorbents into contact with the reaction solution is not limited particularly, and includes a method in which a column is packed with the solid adsorbents through which the reaction solution is allowed to run (hereinafter referred to also as a column method) as well as a method in which a tank is charged with the reaction solution and the solid adsorbents which are agitated thereby accomplish a contact (hereinafter referred to also as a tank method). In view of easy handling upon contact between the solid adsorbents and the reaction solution, the column method is preferred. While the temperature at which the reaction solution and the adsorbents are brought into contact with each other is not limited particularly, it is preferably 10 to 30° C. The phenol present in the reaction solution can be removed, in the column method, due to no adsorption when using a synthetic adsorbent, due to elution at a time different from that for the resveratrol polymerization compound represented by Formulae (2) to (4) shown above when using a soft gel, and by changing the solution composition during elution when using a silica gel.

By purification using the solid adsorbents, it is possible not only to remove the phenol present in the reaction solution but also to desalt and purify the purified material thereby raising the concentration of the resveratrol polymerization compound represented by Formulae (2) to (4).

The method for adjusting the reaction solution to pH7 or below in the Step (b) may, for example, be addition of an acidic substance to the reaction solution. Such an acidic substance is not limited particularly as long as it is a substance capable of making the solution acidic, and may, for example, be hydrochloric acid and citric acid.

The method for precipitating the resveratrol polymerization compound represented by Formulae (2) to (4) in the reaction solution adjusted to pH7 or below may, for example, be employing an aqueous solution whose organic solvent concentration is 10% by volume or less. The organic solvent concentration is preferably 5% or less, and only water is employed in a more preferred case.

The method for recovering the precipitated material is not limited particularly as long as insolubles can be recovered, and may, for example, be filtration and centrifugation.

In the Step (c), the method for heating the reaction solution, the composition obtained by the Step (a), or the aqueous solution containing the composition obtained by the Step (b) is preferably a method in which water vapor is not refluxed. Such a method is not limited particularly, and may, for example, be those employing oil baths, plate-type evaporator, and the like.

The heating condition may be any condition allowing the water contained is evaporated, and it is preferable to employ a temperature of the aqueous solution of 50 to 130° C., preferably, 80 to 100° C., and a treatment period of 10 minutes to 12 hours.

While the treatment of the Steps (a) to (c) enables removal of phenol from the resveratrol heat-treated composition, a substantially complete removal of phenol is possible by further using steam distillation, steam stripping, or using activated charcoal or synthetic adsorbents and the like, thereby obtaining a resveratrol heat-treated composition of a higher quality.

If necessary, drying under reduced pressure or freeze drying is conducted to remove the solvent, thereby obtaining the resveratrol heat-treated composition as a powdery solid.

The resveratrol heat-treated composition thus obtained is not only containing the resveratrol polymerization compound represented by Formulae (2) to (4) shown above but also having a remarkably reduced phenol content. Specifically, the phenol concentration of the methanol solution of the resveratrol heat-treated composition adjusted to a solid content of 20 mg/mL is 100 μg/mL or less, preferably, 50 μg/mL, and more preferably, 5 μg/mL.

The hydroxystilbene cyclization reaction products thus obtained have higher effects which induce differentiation of the white adipocytes into the brown-like adipocytes when compared with that of the starting material hydroxystilbenes, and this brown-like adipocyte-inducing differentiating effect serves to prevent or improve the metabolic syndrome. Accordingly, the agent for inducing differentiation of the white adipocytes into the brown-like adipocytes according to the invention containing as an active ingredient a compound represented by Formula (1) shown above or the composition of this invention containing as an active ingredient a resveratrol polymerization compound represented by Formulae (2) to (4) shown above is useful as a novel prophylactic and/or therapeutic agent against the metabolic syndrome. The effect which induces differentiation of the white adipocytes into the brown-like adipocytes is not found in the hydroxystilbenes as starting materials.

While the agent for inducing differentiation of the white adipocytes into the brown-like adipocytes according to the invention may contain the compound represented by Formula (1) shown above exclusively, it may be a liquid formulation containing the compound represented by Formula (1) shown above as dissolved in a solvent such as ethanol or ethanol-containing aqueous solution, or may be an emulsion or suspension prepared by known methods. While the composition of this invention may contain the compound represented by Formulae (2) to (4) shown above exclusively, it may be a liquid formulation containing the compound represented by Formulae (2) to (4) shown above as dissolved in a solvent such as ethanol or ethanol-containing aqueous solution, or may be an emulsion or suspension prepared by known methods.

The agent for inducing differentiation of the white adipocytes into the brown-like adipocytes according to the invention or the inducing agent composition may contain the hydroxystilbene cyclization reaction products at a level of 0.001% by weight or more.

While the dose of the agent for inducing differentiation of the white adipocytes into the brown-like adipocytes according to the invention or the composition of this invention may vary within a wide range appropriately depending on sex, age, physiological condition, and pathology (obesity level and the like) of the patient, dosage form, administration route, administration frequency, and active ingredient concentration of the drug, the hydroxystilbene cyclization reaction product content may, for example, be about 0.01 to 500 mg/kg, and preferably, about 0.1 to 100 mg/kg a day in an adult. The administration may be once a day, or several times dividedly.

The agent for inducing differentiation of the white adipocytes into the brown-like adipocytes according to the invention or the composition of this invention may be formulated also as a pharmaceutical. The dosage form of this formulation is not limited particularly, and may, for example, be a parenteral formulation such as injection formulation, suppository, eye drop, ointment, aerosol, and the like, or an oral formulation such as tablet, coated tablet, powder, fine granule, granule, capsule, liquid, pill, suspension, emulsion, troche, chewable, syrup, and the like. Those employed in formulating the drugs include pharmaceutically acceptable carrier, excipient, lubricant, binder, disintegrant, diluent, stabilizer, tonicity agent, pH modifier, buffer, and the like.

The carrier and the excipient may, for example, be lactose, sucrose, sodium chloride, glucose, maltose, mannitol, erythritol, xylitol, maltitol, inositol, dextran, sorbitol, albumin, urea, starch, calcium carbonate, kaolin, crystalline cellulose, silicic acid, methyl cellulose, glycerin, sodium alginate, gum arabic, and a mixture thereof.

The lubricant may, for example, be purified talk, stearates, borax, polyethylene glycol, and a mixture thereof.

The binder may, for example, be simple syrup, glucose solution, starch solution, gelatin solution, polyvinyl alcohol, polyvinyl ether, polyvinyl pyrrolidone, carboxymethyl cellulose, shellac, methyl cellulose, ethyl cellulose, water, ethanol, potassium phosphate, and a mixture thereof.

The disintegrant may, for example, be dry starch, sodium alginate, agar powder, laminaran powder, sodium hydrogen carbonate, calcium carbonate, polyoxyethylene sorbitan fatty ester, sodium lauryl sulfate, stearic acid monoglyceride, starch, lactose, and a mixture thereof.

The diluent may, for example, be water, ethyl alcohol, macrogol, propylene glycol, ethoxyisostearyl alcohol, polyoxyisostearyl alcohol, polyoxyethylene sorbitan fatty ester, and a mixture thereof.

The stabilizer may, for example, be sodium pyrosulfite, ethylene diamine tetraacetic acid, thioglycolic acid, thiolactic acid, and a mixture thereof.

The tonicity agent may, for example, be sodium chloride, boric acid, glucose, glycerin, and a mixture thereof.

The pH modifier and the buffer may, for example, be sodium citrate, citric acid, sodium acetate, sodium phosphate, and a mixture thereof.

In addition, the agent for inducing differentiation of the white adipocytes into the brown-like adipocytes according to the invention or the composition of this invention may contain expanding agent, solubilizer, dispersing agent, suspending agent, emulsifier, antioxidant, bacteria inhibitor, colorant, flavor, corrigent, and the like.

Moreover, the agent for inducing differentiation of the white adipocytes into the brown-like adipocytes according to the invention or the composition of this invention may be formulated into a food. The food is not limited particularly, and may, for example, be beverage, alcoholic beverage, jelly, confectionery, functional food, health food, health-oriented food, and the like. In view of storage stability, portability, easy ingestion, and the like, confectioneries are employed preferably, and preferred confectioneries are hard candy, soft candy, gummy candy, tablet, chewing gum, and the like.

When formulating the agent for inducing differentiation of the white adipocytes into the brown-like adipocytes according to the invention or the composition of this invention into a form of a food, the hydroxystilbene cyclization reaction products are contained in the relevant food usually at a level of about 0.001 to 20% by weight.

It is also possible to formulate the agent for inducing differentiation of the white adipocytes into the brown-like adipocytes according to the invention or the composition of this invention into a form of a quasi drug. While the quasi drug is not limited particularly, a nutrient-supplementing quasi drug such as a health drink is preferred. In such a case, the quantity of the hydroxystilbene cyclization reaction products as active ingredients in the quasi drug is usually about 0.001 to 30% by weight.

The agent for inducing differentiation of the white adipocytes into the brown-like adipocytes according to the invention or the composition of this invention can not only be given to humans but also be incorporated into therapeutics or feeds for mammals including rat, mouse, guinea pig, rabbit, sheep, hog, cattle, horse, cat, dog, monkey, chimpanzee, and the like, birds, amphibians, reptiles and the like. The feed may, for example, be livestock feed for sheep, hog, cattle, horse, chicken, and the like, small-sized animal feed for rabbit, rat, mouse, and the like, fish feed for eel, sea bream, yellowtail, shrimp, and the like, pet food for dog, cat, bird, squirrel, and the like.

The present invention is described below in detail based on Examples, which are not intended to restrict the invention. While trans-resveratrol is used as a hydroxystilbene, other hydroxystilbenes can be used for obtaining the compounds by similar reactions.

### EXAMPLES

### Example I-1

### Production and Isolation/Purification of Hydroxystilbene Derivative UHA4003

According to the method described in Example 1 of JP-A No. 2013-28560, production, isolation, and purification of hydroxystilbene derivative UHA4003 were conducted. Thus, 700 mg of trans-resveratrol (manufactured by Tokyo Chemical Industry Co., Ltd.) was dissolved in 14 mL of ethanol, to which 14 mL of 2.5% sodium hydrogen carbonate (manufactured by Wako Pure Chemical Industries, Ltd.) aqueous solution was added to obtain a resveratrol-containing solution (pH9.9). This resveratrol-containing solution was heated using an autoclave (“SANYO LABO AUTOCLAVE” manufactured by SANYO Electric Co., Ltd., same is applied hereinafter) at 130° C. for 20 minutes. Then, the reaction solution obtained by the first autoclave treatment was combined with 14 mL of ethanol and 14 mL of 5.0% sodium hydrogen carbonate aqueous solution, and heated again using the autoclave at 130° C. for 20 minutes. The resultant reaction solution was analyzed by HPLC, which revealed compounds indicated by several peaks.

The HPLC was conducted under the condition shown below.

Column: Reverse phase column “Develosil (trade mark) C-30-UG-5” (4.6 mm i.d.×250 mm)
Mobile phase: A . . . H₂O (0.1% trifluoroacetic acid (TFA)), B . . . acetonitrile (0.1% TFA)
Flow rate: 1 mL/min
Injection volume: 10 μL

### Detection: 254 nm

Gradient (% by volume): 80% A/20% B to 20% A/80% B over 30 minutes, 20% A/80% B to 100% B over 5 minutes, 100% B for 10 minutes (all linear gradient)

Then, several peak compounds were separated and their purities and structures were characterized by HPLC, high resolution FAB-MS (Fast Atom Bombardment-Mass Spectrometry), and nuclear magnetic resonance measurement, based on which a hydroxystilbene derivative (hereinafter designated as UHA4003) was obtained from the peak found near 16 minutes of the elution time under the HPLC analysis condition above.

This UHA4003 was subjected to the nuclear magnetic resonance (NMR) measurement, and the analysis of 1H-NMR, 13C-NMR, and various two-dimensional NMR data indicated that the UHA4003 has a structure represented by Formula (2):

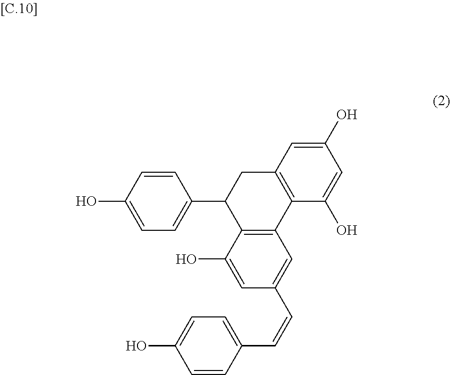 [SMILES: Oc1ccc(/C=C\c2cc(O)c3c(c2)-c2c(O)cc(O)cc2CC3c2ccc(O)cc2)cc1]

which is shown above.

### Example I-2

### Verification of UHA4003's White Adipocytes-to-Brown-Like Adipocyte Differentiation Inducing Effect Indicator Gene Expression

In order to evaluate the white adipocytes-to-brown-like adipocyte differentiation inducing effect, 3T3-L1 cell (mouse-derived precursor adipocyte) was employed in the evaluation. 3T3-L1 precursor adipocyte usually undergoes a differentiation induction process to differentiate into the white adipocyte thereby being matured. Nevertheless, when further induced to differentiate into a brown-like adipocyte, it exhibits a hyperexpression of cell death-inducing DFFA-like effector a (Cidea) gene and CITED1 gene expressed specifically in brown-like adipocytes, an increased expression level of transcription coactivator PGC-1β hyperexpressed in the brown adipocytes and the brown-like adipocytes and cytochrome c oxidase polypeptide 7A1 (Cox7a1) gene expressed in mitochondria, an increased expression level of cytochrome c oxidase protein expressed specifically in mitochondria, and hyperexpression of uncoupling protein (UCP1) gene, any of which is rarely observed in the white adipocytes. In addition, FGF-21 is known, as mentioned in Non-patent Document 6, to be one of the factors which promote the differentiation into the brown-like adipocytes, and it is considered that one of the effects of the hyperexpression of FGF-21 serves to promote the hyperexpression of PGC-1α, which leads to the induction of the white adipocytes into the brown-like adipocytes.

Accordingly, we verified the induction of differentiation of the white adipocytes into the brown-like adipocytes using as an index the expression level of each gene of Cidea, Cox7a1, and UCP1 among the genes above.

Two samples, namely a synthetic PPAR_{Y} agonist rosiglitazone and UHA4003, were employed. Each sample was dissolved in dimethyl sulfoxide (DMSO, manufactured by Wako Pure Chemical Industries, Ltd.) at concentrations of 0.2 mM and 2 mM, and used in the test.

Culture was conducted in Dulbecco's modified Eagle medium (DMEM, trade name, manufactured by Sigma) supplemented with 10% foetal Bovine Serum (FBS, manufactured by Biological industries) and 1% Antibiotic-Antimycotic (manufactured by GIBCO). The adipocytes employed in the test were prepared according to ordinary methods.

The test was conducted as follows. To a 12-well dish for cell culture (manufactured by Corning Incorporated), 1 mL of 3T3-L1 cells were inoculated at a density of 5×10⁴ cells/mL, and cultured for 24 hours under the condition of 37° C. and 5% CO₂. After 24 hours, DMEM medium was replaced with a DMEM medium containing UHA4003 at a final concentration of 20 μM or Rosiglitazone at a final concentration of 1 μM, and the culture was continued until 100% confluent and then for further 48 hours. Then, the medium was replaced with 1 mL of DMEM for differentiation supplemented with 1%, 0.5%, and 0.1% of insulin, dexamethasone, and isobutylmethyl xanthine, respectively, which were attached to “AdipoInducer Reagent” (Trade name, manufactured by Takara Bio Inc.) and also supplemented with each sample at final concentrations of 20 μM and 1 μM, and the differentiation was induced under the condition of 37° C. and 5% CO₂ for 48 hours. After inducing the differentiation for 48 hours, the medium was replaced with 2 mL of DMEM for maintaining culture which was supplemented with 1% insulin, and the culture was conducted further for 1 week to accomplish maturation of the adipocytes. The medium containing only solvent DMSO at 0.5% was employed as a control.

After completion of the culture, an RNA extraction kit (trade name: NucleoSpin (trade mark) RNA II, manufactured by Takara Bio Inc.) was used to extract the total RNA from the cells and purify it. The resultant RNA was subjected to a reverse transcription reaction according to the instruction attached to reverse transcription reagents for 2-step real-time RT-PCR (Trade name, PrimeScript (trade mark) RTMaster Mix, manufactured by Takara Bio Inc.).

Thus, 2 μL of 5×(Primescript RT Master Mix) and 500 ng of the total RNA were mixed, and the entire volume was made 10 μL using RNase Free dH₂O. A thermal cycler for PCR (Trade name: GeneAmp (trade mark) PCR System 9700, manufactured by Applied Biosystem) was employed to conduct the reverse transcription reaction with a program having 1 cycle of “37° C.×15 minutes→85° C.×5 seconds”. The reverse transcription reaction solution was subjected to 5-fold dilution with a diluent for real-time RT-PCR (Trade name: EASY Dilution, manufactured by Takara Bio Inc.), and the resultant diluted solution was employed in real-time RT-PCR analysis.

The real-time RT-PCR analysis was conducted according to an ordinary method. For analysis, “ECO Realtime RT-PCR system” (Trade name, manufactured by Illumina, Inc.) was employed. The primers employed were Cidea forward primer (primer ID: MA104629-F), Cidea reverse primer (primer ID: MA104629-R), Cox7a1 forward primer (primer ID: MA106801-F) and Cox7a1 reverse primer (primer ID: MA106801-R), UCP1 forward primer (primer ID: MA027561-F) and UCP1 reverse primer (primer ID: MA027561-R). The intracellular gene internal standard employed was β-actin, whose primers were ACTB forward primer (primer ID: MA050368-F) and ACTB reverse primer (primer ID: MA050368-R) (8 primers listed above were all manufactured by Takara Bio Inc.)

The reaction employed real-time RT-PCR reagents (Trade name: SYBR (trade mark) Premix EX taqII (Tli RNaseH Plus), manufactured by Takara Bio Inc.) The reaction solution placed in a 48-well PCR plate (manufactured by Illumina, Inc) contained a mixture of 5 μL of 2×(SYBR Premix EX tag II (Tli RNaseH Plus)), 0.08 μL of forward primer (50 μM), 0.08 μL of reverse primer (50 μM), 2 μL of reverse transcription reaction solution, and 2.84 μL of dH₂O (10 μL in total) and subjected to the PCR reaction with a program of “95° C.×30 seconds→‘95° C.×15 seconds→60° C.×1 minute’×40 cycles→95° C.×15 seconds→55° C.×15 seconds→95° C.×15 seconds”.

Based on the Ct values (Threshold Cycle: number of cycles for achieving a certain amplification level (threshold)) of 6-actin and Cidea, Cox7a1 and UCP1 in each cell obtained, the relative value of each gene expression level of Cidea and Cox7a1, UCP1 was calculated. The results are shown in FIG. I-1.

The results indicated, similarly to the time of addition of rosiglitazone, that the gene expression level of Cidea which is the marker gene of the brown-like adipocytes was found to be increased significantly when adding UHA4003, and the expression level of Cox7a1 which is a mitochondria marker gene was also found to be increased significantly. It was also found that the UCP1 gene expression level was also increased significantly. While rosiglitazone was a synthetic agonist, whose effect was higher when comparing with UHA4003, UHA4003 is a beneficial compound capable of being obtained by far more convenient method, and was also indicated to have a possibility of possessing an effect which induces the differentiation of the white adipocytes into the brown-like adipocytes similarly to rosiglitazone.

### Example I-3

### Verification of Expression of Index Gene for Brown-Like Adipocyte-Inducing Differentiating Effect of UHA4003 on Normal Human Subcutaneous Fat

In order to evaluate the white adipocytes-to-brown-like adipocyte differentiation inducing effect observed when using 3T3-L1 cells, human subcutaneous fat-derived normal precursor adipocyte (Lonza Japan) was employed in the evaluation. The normal precursor adipocyte usually undergoes a differentiation into the white adipocyte, but when further induced to differentiate into the brown-like adipocyte, it exhibits UCP-1 gene expression, an increased expression level of Cox7a1 gene which is expressed in mitochondria, and a hyperexpression of CBP/p300-interacting transactivator (CITED1) gene which is expressed specifically in the brown-like adipocytes, any of which is rarely observed in the white adipocytes. Accordingly, we verified the induction of differentiation of the white adipocytes into the brown-like adipocyte using as an index the expression level of each gene of CITED1, Cox7a1, and UCP1.

Two samples, namely a PPAR_{Y} agonist rosiglitazone and UHA4003, were employed. Each sample was dissolved in dimethyl sulfoxide (DMSO, manufactured by Wako Pure Chemical Industries, Ltd.) at appropriate concentrations and used in the test.

The subcutaneous fat-derived normal human precursor adipocytes (manufactured by Lonza Japan) were pre-cultured for 4 days in a precursor adipocyte growth medium (PGM-2) which is a precursor adipocyte basal medium (Preadipocyte Basal Medium-2; PMB-2 medium manufactured by Lonza Japan) supplemented with 10% FBS and 2 mM glutamine, and GA-1000, and then the cells were recovered using an EDTA-trypsin solution and suspended in PGM-2 medium at a density of 8×10⁴ cells/ml, and 0.5 ml aliquots were inoculated onto a 12-well plate for cell culture. After culture in the presence of 5% CO₂ at 37° C. for 24 hours, PGM-2 medium was supplemented with rosiglitazone at a final concentration of 2 μM or with UHA4003 at a final concentration of 40 μM, and 0.5 ml aliquots were further added for culture further for 48 hours. After culture, 1 mL of a medium for differentiation, which is the PGM-2 main differentiation medium (PGM-2 medium supplemented with various factor additives attached to the kit (human insulin, IBMX, dexamethasone, indomethacin)) supplemented with rosiglitazone at a final concentration of 1 μM or with UHA4003 at a final concentration of 20 μM, was added, thereby achieving the adipocyte differentiation and maturation. After this further addition, the culture was continued for 10 days before the test. The medium containing only solvent DMSO at 0.5% was employed as a control.

Similarly to Example I-2, the RNA was extracted and purified, and subjected to the reverse transcription reaction and the real-time RT-PCR. The primers employed were CITED1 forward primer (primer ID: HA204404-F), CITED1 reverse primer (primer ID: HA204404-R), COX7A1 forward primer (primer ID: HA133646-F) and COX7A1 reverse primer (primer ID: HA133646-R), UCP1 forward primer (primer ID: HA158451-F) and UCP1 reverse primer (primer ID: HA158451-R). The intracellular gene internal standard employed was β-actin, whose primers were ACTB forward primer (primer ID: HA067803-F) and ACTB reverse primer (primer ID: HA067803-R) (8 primers listed above were all manufactured by Takara Bio Inc.).

Based on the Ct values (Threshold Cycle: number of cycles for achieving a certain amplification level (threshold)) of 6-actin, CITED1, COX7A1, and UCP1 in each cell obtained, the relative value of each gene expression level of CITED1, COX7A1, and UCP1 was calculated. The results are shown in FIG. I-2.

The results indicated, similarly to the time of addition of rosiglitazone, that the expression level of CITED1 gene which is the marker gene of the brown-like adipocytes was found to be increased significantly when adding UHA4003, and it was further revealed that the expression level of COX7A1 which is a mitochondria marker gene was increased significantly. It was also found that the UCP1 gene expression level was also increased significantly. It indicates that the human subcutaneous fat-derived normal precursor adipocyte, which is differentiated usually into a white adipocyte, was differentiated this time into the brown-like adipocyte in the presence of UHA4003. Thus, it was indicated that UHA4003 has an extremely potent effect which induces the differentiation of the white adipocytes into the brown-like adipocytes.

Based on the results observed in Examples I-2 and I-3 described above, addition of UHA4003 resulted in a significant increase in the intracellular mitochondria level and marked induction of the differentiation of the white adipocytes into the brown-like adipocytes, which indicate that UHA4003 has an excellent effect which induces the differentiation into the brown-like adipocytes.

UHA4003 is excellent also in terms of inexpensive preparation from food constituents in a single step when compared with rosiglitazone which is a synthetic agonist posing a high production cost.

(Example II-1: Production and purification of resveratrol polymerization compound-containing composition)

Production and purification of a resveratrol polymerization compound-containing composition were conducted by the procedure shown below. One gram of trans-resveratrol (manufactured by Techno Science Co., Ltd.) was dissolved in 10 mL of ethanol, to which 10 mL of 10% sodium hydrogen carbonate (manufactured by Wako Pure Chemical Industries, Ltd.) aqueous solution was added to obtain a resveratrol-containing solution (pH9.9). This resveratrol-containing solution was heated in an autoclave at 130° C. for 90 minutes to obtain a resveratrol polymerization compound-containing solution.

Then, the resveratrol polymerization compound-containing solution was diluted and dissolved in 1 L of distilled water and loaded onto 400 g of a synthetic adsorbent DIAION (trade mark) HP-20 (manufactured by Mitsubishi Chemical Corporation). After washing with 1 L of distilled water, elution was conducted using 1 L of 100% ethanol. The solvent was removed by drying under reduced pressure to obtain 200 mg of a resveratrol polymerization compound-containing composition (hereinafter referred to as a derivative-containing composition). The resultant polymerization compound composition was dissolved in methanol at a concentration of 2 mg/mL, and a 10 μL aliquot was analyzed by HPLC.

The HPLC analysis was conducted under the condition shown below.

Column: CAPCELL PAK UG80 column (4.6 mm I.D.×250 mm, manufactured by Shiseido Co., Ltd.)
Mobile phaseA: 0.1% trifluoroacetic acid (TFA, manufactured by Wako Pure Chemical Industries, Ltd.)/H₂O
Mobile phaseB: 0.1% TFA/acetonitrile (manufactured by Wako Pure Chemical Industries, Ltd.)
Gradient (% by volume): 100% A/0% B to 0% A/100% B over 33 minutes, 0% A/100% B for 7 minutes (all linear gradient)

The chromatograms obtained are shown in FIG. II-1. The reaction solution obtained was analyzed by HPLC, which detected several peaks different from the peak of resveratrol, thus giving a composition containing several compounds produced.

Then, the resultant reaction products were subjected to preparative HPLC to isolate the compounds contained in the peaks indicated by (1), (2), and (3) in FIG. II-1, and dried by a standard method to obtain the compounds contained in the peaks indicated by (1) and (2) as pale tan powders and the compounds contained in the peak indicated by (3) as a tan powder.

Then the molecular weights of the three compounds above were measured according to the method described in Example 2 of JP-A No. 2011-251914 using a high resolution Negative-FAB-MS (Fast Atom Bombardment-Mass Spectrometry) and nuclear magnetic resonance (NMR) measurement was also conducted, and the results revealed that the compound contained in peak (1) is the compound represented by Formula (3) shown above (hereinafter referred to as UHA4002) while the compound contained in peak (2) is the compound represented by Formula (2) shown above (hereinafter referred to as UHA4003).

On the other hand, the compound contained in peak (3) is the compound which is a novel compound which was not reported before.

Thus, the value found by the high resolution Negative-FAB-MS was 647.2144, which was compared with the calculated value, and the following molecular formula was obtained.

Calculated as C₄₂H₃₁O₇(M-H⁻): 647.2148

Molecular formula: C₄₂H₃₂O₇

The nuclear magnetic resonance (NMR) measurement was then conducted, and, based on the analysis of ¹H-NMR, ¹³C-NMR, and various two-dimensional NMR data, it was confirmed that the compound contained in peak (3) described above was a novel compound having a structure represented by Formula (4) shown above (hereinafter referred to as UHA4022).

The data found in the NMR for UHA4022 represented as follows:

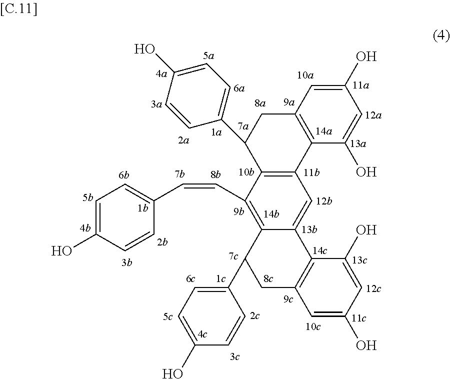 [SMILES: Oc1ccc(/C=C\c2c3c(cc4c2C(c2ccc(O)cc2)Cc2cc(O)cc(O)c2-4)-c2c(O)cc(O)cc2CC3c2ccc(O)cc2)cc1]

are ¹H nuclear magnetic resonance spectrum and ¹³C nuclear magnetic resonance spectrum shown in Table 1.

The values are indicated in δ and ppm, and the solvent employed was DMSO-d₆.

TABLE 1

| | UHA4022 | |
|---|---|---|
| | ¹³C | ¹H |
| 1a | 133.4 | |
| 2a, 6a | 128.5 | 6.79 (2H, d, J = 8.9 Hz) |
| 3a, 5a | 114.6 | 6.48 (2H, d, J = 8.9 Hz) |
| 4a | 155.2 | |
| 7a | 37.8 | 4.73 (1H, d, J = 6.0 Hz) |
| 8a | 38.3 | 2.94 (1H, d, J = 13.6 Hz), |
| | | 3.13 (1H, dd, J = 6.0, 13.6 Hz) |
| 9a | 138.0 | |
| 10a | 107.5 | 6.01 (1H, d, J = 1.8 Hz) |
| 11a | 157.0 | |
| 12a | 102.0 | 6.27 (1H, d, J = 1.8 Hz) |
| 13a | 155.6 | |
| 14a | 112.9 | |
| 1b | 132.2 | |
| 2b, 6b | 128.4 | 7.05 (2H, d, J = 9.0 Hz) |
| 3b, 5b | 114.9 | 6.60 (2H, d, J = 9.0 Hz) |
| 4b | 155.2 | |
| 7b | 122.3 | 7.75 (1H, d, J = 9.6 Hz) |
| 8b | 119.3 | 7.59 (1H, d, J = 9.6 Hz) |
| 9b | 131.0 | |
| 10b | 124.8 | |
| 11b | 132.6 | |
| 12b | 112.7 | 8.27 (1H, s) |
| 13b | 139.5 | |
| 14b | 117.2 | |
| 1c | 135.6 | |
| 2c, 6c | 128.4 | 7.04 (2H, d, J = 9.0 Hz) |
| 3c, 5c | 114.9 | 6.61 (2H, d, J = 9.0 Hz) |
| 4c | 155.2 | |
| 7c | 50.0 | 4.17 (1H, t, J = 7.8 Hz) |
| 8c | 39.2 | 3.56 (2H, ddd-like) |
| 9c | 137.1 | |
| 10c | 111.0 | 9.05 (1H, d, J = 2.4 Hz) |
| 11c | 154.3 | |
| 12c | 117.5 | 6.63 (1H, d, J = 2.4 Hz) |
| 13c | 154.0 | |
| 14c | 122.9 | |

The physicochemical characteristics of UHA4022 are as follows.

### (Appearance)

Tan powder

### (Solubility)

Water: Hardly soluble

### Methanol: Soluble

### Ethanol: Soluble

### DMSO: Soluble

Chloroform: Hardly soluble
Ethyl acetate: Hardly soluble

### Example II-2

### Verification of Expression of Index Gene for White Adipocytes-to-Brown-Like Adipocyte Differentiation Inducing Effect of Derivative-Containing Composition

In order to evaluate the white adipocytes-to-brown-like adipocyte differentiation inducing effect, 3T3-L1 cell (mouse-derived precursor adipocyte) was used for evaluation similarly to Example I-2. In this Example, the induction of differentiation of the white adipocytes into the brown-like adipocyte was verified using as an index the expression level of each gene of FGF-21, PGC-1α, and Cox7a1.

Two samples, namely a synthetic PPAR_{Y} agonist rosiglitazone and the derivative-containing composition prepared in Example II-1 were employed. Each sample was dissolved in dimethyl sulfoxide (DMSO, manufactured by Wako Pure Chemical Industries, Ltd.) at concentrations of 0.2 mM and 20 mg/mL and used in the test.

Culture was conducted in Dulbecco's modified Eagle medium supplemented with 10% foetal Bovine Serum and 1% Antibiotic-Antimycotic. The adipocytes employed in the test were prepared according to ordinary methods.

The test was conducted as follows. To a 12-well dish for cell culture (manufactured by Corning Incorporated), 1 mL of 3T3-L1 cells were inoculated at a density of 5×10⁴ cells/mL, and cultured for 24 hours under the condition of 37° C. and 5% CO₂. After 24 hours, DMEM medium was replaced with a DMEM medium containing the composition at a final concentration of 20 μM or Rosiglitazone at a final concentration of 1 μM, and the culture was continued until 100% confluent and then for further 48 hours. Then, the medium was replaced with 1 mL of DMEM for differentiation supplemented with 1%, 0.5%, and 0.1% of insulin, dexamethasone, and isobutylmethyl xanthine, respectively, which were attached to “AdipoInducer Reagent” and also supplemented with each sample at final concentrations of 20 μg/mL and 1 μM, and the differentiation was induced under the condition of 37° C. and 5% CO₂ for 48 hours. The medium containing only solvent DMSO at 0.5% was employed as a control.

After completion of the culture, the RNA extraction kit was used similarly to Example I-2 to extract the total RNA from the cells and purify it, and the resultant RNA was subjected to a reverse transcription reaction according to the instruction attached to reverse transcription reagents for 2-step real-time RT-PCR.

The reverse transcription reaction solution was then subjected to 5-fold dilution with a diluent for real-time RT-PCR, and the resultant diluted solution was employed in real-time RT-PCR analysis.

The real-time RT-PCR analysis was conducted according to an ordinary method. For analysis, “ECO Real-time RT-PCR system” (Trade name, manufactured by Illumina, Inc.) was employed. The primers employed were FGF-21 forward primer (primer ID: MA-126886-F), FGF-21 reverse primer (primer ID: MA-126886-R), PGC-1α forward primer (primer ID: MA-114509-F), PGC-1α reverse primer (primer ID: MA-114509-R), Cox7a1 forward primer (primer ID: MA106801-F), and Cox7a1 reverse primer (primer ID: MA106801-R). The intracellular gene internal standard employed was β-actin, whose primers were ACTB forward primer (primer ID: MA050368-F) and ACTB reverse primer (primer ID: MA050368-R) (8 primers listed above were all manufactured by Takara Bio Inc.)

The reaction employed real-time RT-PCR reagents (Trade name: SYBR (trade mark) Select Master Mix, manufactured by Life Technologies). The reaction solution placed in a 48-well PCR plate (manufactured by Illumina, Inc.) containing a mixture of 5 μL of 2×(SYBR Select Master Mix), 0.08 μL of forward primer (50 μM), 0.08 μL of reverse primer (50 μM), 2 μL of reverse transcription reaction solution, and 2.84 μL of dH₂O (10 μL in total), and subjected to the PCR reaction with a program of “50° C.×2 minutes→95° C.×2 minutes→‘95° C.×15 seconds→60° C.×1 minute’×40 cycles→95° C.×15 seconds→55° C.×15 seconds→95° C.×15 seconds”.

Based on the Ct values (Threshold Cycle: number of cycles for achieving a certain amplification level (threshold)) of 6-actin, FGF-21, PGC-1α, and Cox7a1 in each cell obtained, the relative value of each gene expression level of FGF-21, PGC-1α, and Cox7a1 was calculated. The results are shown in FIG. II-2.

The results indicated, similarly to the time of addition of rosiglitazone, that addition of the derivative-containing composition resulted in a significant increase in the expression level of FGF-21 gene which is one of the brown-like adipocyte inducing factor together with PGC-1α gene in response to the expression of FGF-21 gene, and it also became evident that the expression level of Cox7a1 which is a mitochondria marker gene was increased significantly. Rosiglitazone is a synthetic agonist and has a higher effect when compared with the derivative-containing composition. Nevertheless, a thiazolidine-based compound including rosiglitazone has a potent side effect on humans such as edema and cardiac insufficiency and is a compound which is problematic in terms of safety. On the other hand, the derivative-containing composition is a beneficial composition capable of being obtained by far more convenient method, and its precursor compound resveratrol is a compound which occurs widely in nature and extremely safe, and hydroxystilbenes including resveratrol as well as their polymerization products have been ingested for a long time because of their wide distribution in nature. Accordingly, the inventive product was proven not only to be highly safe but also to be a useful composition which is highly possible to possess an extremely potent effect which induces the differentiation of the white adipocytes into the brown-like adipocytes similarly to rosiglitazone.

### Example II-3

### Verification of Expression of Index Gene for White Adipocytes-to-Brown-Like Adipocyte Differentiation Inducing Effect of Derivative-Containing Composition

Since it was evaluated in Example II-2 whether the derivative-containing composition induces the differentiation from the white adipocytes into the brown-like adipocytes using the cells immediately after the induction of the differentiation, it was evaluated in Example II-3 whether the relevant induced cell maintains the trait of the brown-like adipocytes even after maturation using matured adipocytes. The evaluation was made using as indexes the expression level of each gene of FGF-21, PGC-1α, and cell death-inducing DFFA-like effector a (Cidea) which is a brown-like adipocyte marker gene as well as the expression level of USP1 gene when adding adrenaline to the adipocyte after maturation based on the understanding that the brown-like adipocyte responds to the adrenaline stimulation thereby increasing the expression level of UCP1 gene, whereby the induction of the differentiation of the white adipocytes into the brown-like adipocytes was confirmed.

By the method similar to that in Example II-2, the same compositions and compounds were added to induce the differentiation of the 3T3-L1 precursor adipocyte. After inducing the differentiation for 48 hours, the medium was replaced with 2 mL of DMEM for maintaining culture supplemented with 1% insulin, and further cultured for 1 week thereby achieving the maturation of the adipocyte. The adrenaline stimulation was given to the matured cells by replacement with 2 mL of DMEM for maintaining culture supplemented with 1 μM of L-adrenaline (manufactured by Wako Pure Chemical Industries, Ltd.), which was cultured for 2 hours and the response was verified.

After maturation for 1 week, or after the adrenaline stimulation, the method similar to that in Example II-2 was employed for the extraction of the total RNA, purification, and reverse transcription reaction, and the real-time RT-PCR analysis was conducted. The primers employed in the analysis were same as those employed in Example II-2 for each gene of FGF-21, PGC-1α, and ACTB, and PGC-1β forward primer (primer ID: MA125505-F) and PGC-1β reverse primer (primer ID: MA125505-R) for PGC-1β gene, and Cidea forward primer (primer ID: MA104629-F) and Cidea reverse primer (primer ID: MA104629-R) for Cidea gene, and UCP1 forward primer (primer ID: MA027561-F) and UCP1 reverse primer (primer ID: MA027561-R) for UCP1 gene (all manufactured by Takara Bio Inc.).

Based on the Ct values (Threshold Cycle: number of cycles for achieving a certain amplification level (threshold)) of 6-actin, FGF-21, PGC-1α, PGC-1β, Cidea, and UCP1 in each cell obtained, the relative value of each gene expression level of FGF-21, PGC-1β, PGC-1α, Cidea, and UCP1 was calculated. The results are shown in FIG. II-3.

The results indicated, similarly to the time of addition of rosiglitazone, a significant increase in the expression level of FGF-21 gene which is one of the brown-like adipocyte inducing factor together with PGC-1α gene in response to the expression of FGF-21 gene was observed, and it also became evident that the expression level of Cidea which is a brown-like adipocyte marker gene and the expression level of PGC-1β whose expression is increased in the brown adipocytes were increased significantly. In addition, the UCP1 gene expression-increasing effect of adrenaline, when comparing between the presence and absence of adrenaline, became greater by 3.1 times in the control in the presence of adrenaline, while that in the presence of rosiglitazone became greater by 6.2 times and that in the presence of the derivative-containing composition of the invention became greater by 6.7 times, showing a substantial increase in the expression, which is characteristic only to the brown-like adipocytes. In other words, it was revealed that by adding the composition of this invention, 3T3-L1 precursor adipocytes are induced to differentiate into the brown-like adipocytes and maintains the trait of the brown-like adipocytes even after maturation, thus indicating that the composition of this invention is highly possible to possess an extremely potent effect which induces the differentiation of the white adipocytes into the brown-like adipocytes.

### Example II-4

### Verification of Expression of Index Gene for Brown-Like Adipocyte-Inducing Differentiating Effect of Derivative-Containing Composition on Normal Human Subcutaneous Fat

In order to evaluate the white adipocytes-to-brown-like adipocyte differentiation inducing effect observed when using 3T3-L1 cells, human subcutaneous fat-derived normal precursor adipocyte (Lonza Japan) was employed similarly to Example I-3 to confirm the induction of the differentiation of the white adipocytes into the brown-like adipocytes using as an index the expression level of each gene of CITED1, Cox7a1, and UCP1.

Two samples, namely a PPAR_{Y} agonist rosiglitazone and the derivative-containing composition, were employed. Each sample was dissolved in dimethyl sulfoxide (DMSO, manufactured by Wako Pure Chemical Industries, Ltd.) at appropriated concentrations and used in the test.

The culture of the subcutaneous fat-derived normal human precursor adipocyte (manufactured by Lonza Japan) was conducted similarly to Example I-3 except for changing the sample.

Similarly to Example II-2, the RNA was extracted and purified, and subjected to the reverse transcription reaction and the real-time RT-PCR. The primers employed were CITED1 forward primer (primer ID: HA204404-F), CITED1 reverse primer (primer ID: HA204404-R), PGC-1β forward primer (primer ID: HA172103-F), PGC-1β reverse primer (primer ID: HA172103-R), COX7A1 forward primer (primer ID: HA133646-F) and COX7A1 reverse primer (primer ID: HA133646-R), UCP1 forward primer (primer ID: HA158451-F) and UCP1 reverse primer (primer ID: HA158451-R). The intracellular gene internal standard employed was β-actin, whose primers were ACTB forward primer (primer ID: HA067803-F) and ACTB reverse primer (primer ID: HA067803-R) (10 primers listed above were all manufactured by Takara Bio Inc.).

Based on the Ct values of 6-actin, CITED1, PGC-1β, COX7A1, and UCP1 in each cell obtained, the relative value of each gene expression level of CITED1, PGC-1β, COX7A1, and UCP1 was calculated. The results are shown in FIG. II-4.

The results indicated, similarly to the time of addition of rosiglitazone, that the expression level of CITED1 gene which is the marker gene of the brown-like adipocytes was found to be increased significantly when adding the derivative-containing composition, and it was further revealed that the expression level of COX7A1 which is a mitochondria marker gene was increased significantly. It was also found that the expression level of each gene of UCP1 gene and PGC-1β was also increased significantly. It indicates that the human subcutaneous fat-derived normal precursor adipocyte, which is differentiated usually into a white adipocyte, was differentiated this time into the brown-like adipocyte in the presence of the derivative-containing composition. Thus, it was indicated that the derivative-containing composition of the invention has an extremely potent effect which induces the differentiation of the white adipocytes into the brown-like adipocytes.

Based on the results observed in Example II-2, II-3, and II-4 described above, addition of the derivative-containing composition resulted in a significant increase in the intracellular mitochondria level and marked induction of the differentiation of the white adipocytes into the brown-like adipocytes, which indicate that the derivative-containing composition has an excellent effect which induces the differentiation into the brown-like adipocytes.

The derivative-containing composition is excellent also in terms of inexpensive preparation from food constituents in a single step when compared with rosiglitazone which is a synthetic agonist posing a high production cost.

### Example II-5

### Pharmaceutical Containing Composition of Invention

One gram of the derivative-containing composition obtained similarly to Example II-1 was dissolved in ethanol, and the resultant solution was allowed to be adsorbed onto microcrystalline cellulose, which was then dried under reduced pressure. Ten parts of the adsorbed inventive material, 23 parts of corn starch, 12 parts of lactose, 8 parts of carboxymethylcellulose, 32 parts of microcrystalline cellulose, 4 parts of polyvinyl pyrrolidone, 3 parts of magnesium stearate, and 8 parts of talc were mixed and compacted into tablets, thereby obtaining the tablet formulation containing the material of the present invention.

### Example II-6

### Quasi Drug Containing Composition of Invention

Ten milliliters of an ethanol solution obtained by dissolving 1.2 g of the derivative-containing composition obtained similarly to Example II-1 in ethanol, 20 g of taurine, 0.12 g of vitamin B1 nitrate, 0.6 g of sodium benzoate, 4 g of citric acid, 60 g of sugar, and 10 g of polyvinyl pyrrolidone were dissolved in purified water to make the entire volume 1000 mL. The pH was adjusted to 3.2 using dilute hydrochloric acid. From 1000 mL of the resultant solution, a 50 mL aliquot was filled in a glass bottle, which was sterilized at 80° C. for 30 minutes to obtain a health drink as a quasi drug.

### Example III-1

### Production and Purification of Resveratrol Polymerization Compound-Containing Composition

Production and purification of the resveratrol polymerization compound-containing composition were conducted in the procedure described below. Forty grams of trans-resveratrol (manufactured by Techno Science Co., Ltd.) was combined with 400 mL of 10% sodium carbonate (manufactured by Wako Pure Chemical Industries, Ltd.) aqueous solution to obtain a resveratrol-containing aqueous solution (pH9.9). This resveratrol-containing solution was heated in a pressure cooker (ZERO minute Pressure Cooker, Asahi Lightmetal Industry Co., Ltd.) at 128° C. for 120 minutes to obtain a resveratrol polymerization compound-containing solution (Comparative Product 1).

Then, starting from Comparative Product 1, the following Prototype Products 1 to 4 were prepared.

The entire amount of Comparative Product 1 was loaded onto 4.0 kg of a synthetic adsorbent DIAION (trade mark) HP-20 (manufactured by Mitsubishi Chemical Corporation). After washing with 24 L of distilled water, 16 L of 100% ethanol was used for elution. The solvent was evaporated off by drying under reduced pressure to obtain 22 g of a resveratrol polymerization compound-containing composition (hereinafter referred to as a derivative-containing composition) (Prototype Product 1).

On the other hand, Comparative Product 1 was combined with an appropriate amount of concentrated hydrochloric acid (manufactured by Wako Pure Chemical Industries, Ltd.) and the solution was adjusted to pH7 or below while validating with pH test paper, and water-insoluble components were recovered by filtration to obtain 30 g (Prototype Product 2).

In addition, Comparative Product 1 was combined with an appropriate amount of concentrated hydrochloric acid and the solution was adjusted to pH7 or below while validating with pH test paper, and the material thus adjusted was suspended in 3 L of distilled water, and then the derivative-containing composition was kept at 85° C. for 2 hours using an oil bath (manufactured by AS ONE Corporation), which procedure was repeated twice, thereby obtaining a derivative composition (Prototype Product 3).

Furthermore, Prototype Product 1 was suspended in 3 L of distilled water, and then the derivative-containing composition was kept at 85° C. for 2 hours using an oil bath, which procedure was repeated twice, thereby obtaining a derivative composition (Prototype Product 4).

The resultant Comparative Product 1 and Prototype Products 1 to 4 were dissolved in methanol at a concentration of 2 mg/mL, and 10 μL aliquots were analyzed by HPLC similarly to Example II-1.

Among the chromatograms obtained, the chart of Prototype Product 4 was shown as a representative in FIG. III-1. The reaction solution obtained was analyzed by HPLC, which detected several peaks different from the peak of resveratrol, thus giving a composition containing several compounds produced.

Then, the resultant reaction products were subjected to preparative HPLC to isolate the compounds contained in the peaks indicated by (1), (2), and (3) in FIG. III-1, and dried by a standard method to obtain the compounds contained in the peaks indicated by (1) and (2) as pale tan powders and the compounds contained in the peak indicated by (3) as a tan powder.

Then the molecular weights of the three compounds above were measured according to the method described in Example 2 of JP-A No. 2011-251914 using a high resolution Negative-FAB-MS and NMR measurement was also conducted. The results revealed that the compound contained in peak (1) is the compound represented by Formula (3) shown above (hereinafter referred to as UHA4002), that the compound contained in peak (2) is the compound represented by Formula (2) shown above (hereinafter referred to as UHA4003), and that the compound contained in peak (3) is the compound represented by Formula (4) shown above (hereinafter referred to as UHA4022).

### Experimental Example III-1

### Measurement of Phenol Concentration of Resveratrol Polymerization Compound-Containing Composition

The concentration of phenol contained in Comparative Product 1 and Prototype Products 1 to 4 prepared in Example III-1 was measured using a gas chromatograph mass spectrometer. Each of Comparative Product 1 and Prototype Products 1 to 4 was dissolved in methanol at a concentration of 20 mg/mL, and an 1 mL aliquot was subjected to the gas chromatograph mass spectrometer (manufactured by JMS-Q1000MC, JEOL Ltd.) to analyze and compare the phenol contents.

The gas chromatograph mass spectrometry was conducted under the following conditions. The instruments employed were Rtx (trade mark)-200MS (0.25 mm id×30 m, film thickness of 0.25 μm, manufactured by Restek Corporation), SPME fiber (50/30 μm, DVB/CAR/PDMS, manufactured by Supelco), a GC-MS of Model JMS-Q1000GC K9 (manufactured by JEOL Ltd.). The SPME fiber conditions were preheating (65° C., 1 minute), adsorption time (30 minutes), desorption time (10 minutes), injection temperature (250° C.). The GS was conducted with a temperature condition (50° C. for 10 minutes→10° C./minute to 320° C.→320° C. for 10 minutes), a split ratio of 20, and helium as a carrier gas.

Phenol (manufactured by Wako Pure Chemical Industries, Ltd.) was employed to obtain a calibration curve to determine the phenol concentration of each Prototype Product and Comparative Product, and the phenol content per 20.0 mg/mL solid was determined. The results are shown in Table 2.

TABLE 2

| Sample | Phenol concentration (μg/mL) |
|---|---|
| Comparative Product 1 | 1041 |
| Prototype Product 1 | 47.4 |
| Prototype Product 2 | 52.3 |
| Prototype Product 3 | 53.6 |
| Prototype Product 4 | 0.66 |

Based on the results of the measurement, each Prototype Product had a phenol concentration not higher than 1/20 of that of Comparative Product 1 obtained immediately after production by the pressure cooker, which was as low as 1/1500 or less in case of Prototype Product 4 which was subjected to the step twice. Accordingly, the inventive products, such as Prototype Product 1 to 4, were revealed to be the products in which the phenol contained in the inventive product, whose presence was missed in conventional products, was reduced to an extremely low level.

### Example III-2

### Verification of Expression of Index Gene for White Adipocytes-to-Brown-Like Adipocyte Differentiation Inducing Effect of Derivative-Containing Composition

In order to evaluate the white adipocytes-to-brown-like adipocyte differentiation inducing effect, 3T3-L1 cell (mouse-derived precursor adipocyte) was used for evaluation similarly to Example II-2. In this Example, we verified the induction of differentiation of the white adipocytes into the brown-like adipocyte using as an index the expression level of each gene of FGF-21, PGC-1α, PGC-1β, and CITED1.

Two samples, namely a synthetic PPAR_{Y} agonist rosiglitazone and Prototype Product 4 obtained in Example III-1 were employed. Each sample was dissolved in dimethyl sulfoxide (DMSO, manufactured by Wako Pure Chemical Industries, Ltd.) at concentrations of 0.2 mM and 20 mg/mL and used in the test.

Culture was conducted in Dulbecco's modified Eagle medium supplemented with 10% foetal Bovine Serum and 1% Antibiotic-Antimycotic. The adipocytes employed in the test were prepared according to ordinary methods.

The test was conducted similarly to Example II-2 except for changing the sample.

After completion of the culture of 3T3-LT cells, the RNA extraction kit was used similarly to Example II-2 to extract the total RNA from the cells and purify it, and the resultant RNA was subjected to a reverse transcription reaction according to the instruction attached to reverse transcription reagents for 2-step real-time RT-PCR.

The reverse transcription reaction solution obtained was then subjected to 5-fold dilution with a diluent for real-time RT-PCR, and the resultant diluted solution was employed in real-time RT-PCR analysis.

The real-time RT-PCR analysis was conducted according to an ordinary method. For analysis, “ECO Real-time RT-PCR system” (Trade name, manufactured by Illumina, Inc.) was employed. The primers employed were FGF-21 forward primer (primer ID: MA-126886-F), FGF-21 reverse primer (primer ID: MA-126886-R), PGC-1α forward primer (primer ID: MA-114509-F), PGC-1α reverse primer (primer ID: MA-114509-R), PGC-1β forward primer (primer ID: MA125505-F), PGC-1β reverse primer (primer ID: MA125505-R), CITED1 forward primer (primer ID: MA104748-F), CITED1 reverse primer (primer ID: MA104748-R). The intracellular gene internal standard employed was 6-actin, whose primers were ACTB forward primer (primer ID: MA050368-F) and ACTB reverse primer (primer ID: MA050368-R) (8 primers listed above were all manufactured by Takara Bio Inc.)

The PCR reaction and the program were similar to those in Example II-2.

Based on the Ct values β-actin, FGF-21, PGC-1α, PGC-1β, and CITED1 in each cell obtained, the relative value of each gene expression level of FGF-21, PGC-1α, PGC-1β, and CITED1 was calculated. The results are shown in FIG. III-2.

The results indicated, similarly to the time of addition of rosiglitazone, that addition of the derivative-containing composition resulted in a significant increase in the expression level of FGF-21 gene which is one of the brown-like adipocyte inducing factor (left top figure) together with PGC-1α gene (right top figure) in response to the expression of FGF-21 gene, and it also became evident that the expression level of Cited1 which is the marker gene of the brown-like adipocytes (right bottom figure) and the expression level of PGC-1β (left bottom figure) were increased significantly. Rosiglitazone is a synthetic agonist and has a higher effect when compared with the derivative-containing composition. Nevertheless, a thiazolidine-based compound including rosiglitazone has a potent side effect on humans such as edema and cardiac insufficiency and is a compound which is problematic in terms of safety. On the other hand, the derivative-containing composition is a beneficial composition capable of being obtained by far more convenient method, and its precursor compound resveratrol is a compound which occurs widely in nature and extremely safe, and hydroxystilbenes including resveratrol as well as their polymerization products have been ingested for a long time because of their wide distribution in nature. Accordingly, the inventive product was proven not only to be highly safe but also to be a useful composition which is highly possible to possess an extremely potent effect which induce the differentiation of the white adipocytes into the brown-like adipocytes similarly to rosiglitazone.

Based on the results observed in Example III-2 described above, addition of the derivative-containing composition resulted in a significant increase in the intracellular mitochondria level and marked induction of the differentiation of the white adipocytes into the brown-like adipocytes, which indicate that the derivative-containing composition has an excellent effect which induce the differentiation into the brown-like adipocytes.

The derivative-containing composition is excellent also in terms of inexpensive preparation from food constituents in a single step when compared with rosiglitazone which is a synthetic agonist posing a high production cost.

### Example III-3

### Pharmaceutical Containing Composition of Invention

One gram of the derivative-containing composition obtained similarly to Example III-1 was dissolved in ethanol, and the resultant solution was allowed to be adsorbed onto microcrystalline cellulose, which was then dried under reduced pressure. Ten parts of the adsorbed inventive material, 23 parts of corn starch, 12 parts of lactose, 8 parts of carboxycellulose, 32 parts of microcrystalline cellulose, 4 parts of polyvinyl pyrrolidone, 3 parts of magnesium stearate, and 8 parts of talc were mixed and compacted into tablets, thereby obtaining the tablet formulation containing the material of the present invention.

### Example III-4

### Quasi Drug Containing Composition of Invention

Ten milliliters of an ethanol solution obtained by dissolving 1.2 g of the derivative-containing composition obtained similarly to Example III-1 in ethanol, 20 g of taurine, 0.12 g of vitamin B1 nitrate, 0.6 g of sodium benzoate, 4 g of citric acid, 60 g of sugar, and 10 g of polyvinyl pyrrolidone were dissolved in purified water to make the entire volume 1000 mL. The pH was adjusted to 3.2 using dilute hydrochloric acid. From 1000 mL of the resultant solution, a 50 mL aliquot was filled in a glass bottle, which was sterilized at 80° C. for 30 minutes, to obtain a health drink as a quasi drug.

### Example III-5

### Food Containing Composition of this Invention

One gram of the derivative-containing composition obtained similarly to Example III-1 was dissolved preliminarily in 100 mL of ethanol, in which 500 g of sugar and 400 g of starch syrup were mixed and dissolved and combined with 100 g of fresh cream, 20 g of butter, 70 g of condensed milk, and 1.0 g of emulsifier, and placed in a vacuum pan to effect −550 mmHg pressure reduction, and concentrated at 115° C. to obtain a milk hard candy whose water content was 3.0% by weight. This milk hard candy is not only a palatable confectionery but also a functional food which reduces the differentiation of precursor adipocyte into mature adipocytes thereby being hopeful to serve for prevention of obesity.

1. An agent for inducing differentiation of white adipocytes into brown-like adipocytes comprising a compound which is a cyclization reaction product of a hydroxystilbene and which is represented by Formula (1): 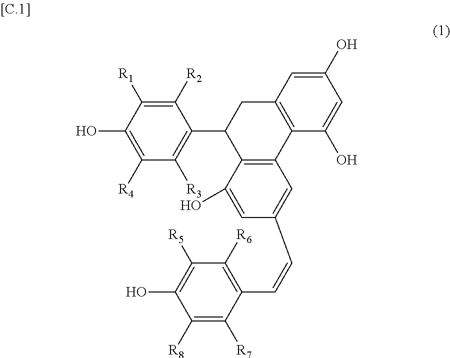
(wherein all of R₁ to R₈ are hydrogen atoms), or a pharmaceutically acceptable salt thereof.

2. The agent for inducing differentiation of white adipocytes into brown-like adipocytes according to claim 1 wherein the hydroxystilbene is resveratrol.

3. (canceled)

4. A composition of an agent for inducing differentiation of white adipocytes into brown-like adipocytes which is a composition obtained by heating resveratrol under an alkaline condition and which comprises three resveratrol polymerization compounds represented by Formula (2): 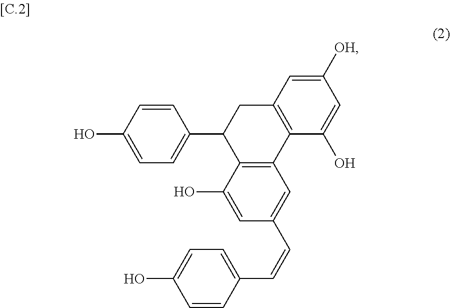 [SMILES: Oc1ccc(/C=C\c2cc(O)c3c(c2)-c2c(O)cc(O)cc2CC3c2ccc(O)cc2)cc1]
Formula (3): 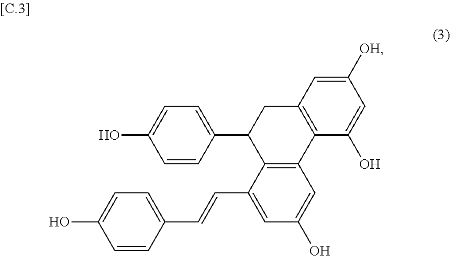 [SMILES: Oc1ccc(/C=C/c2cc(O)cc3c2C(c2ccc(O)cc2)Cc2cc(O)cc(O)c2-3)cc1]
and Formula (4):

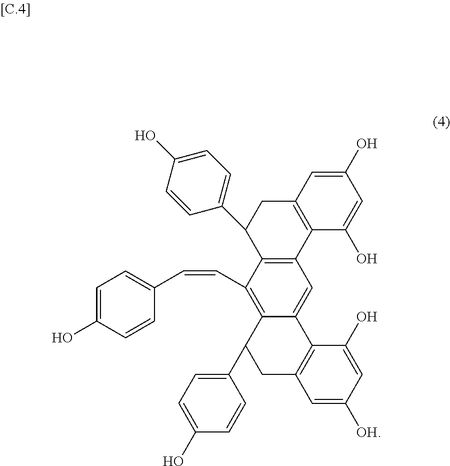 [SMILES: Oc1ccc(/C=C\c2c3c(cc4c2C(c2ccc(O)cc2)Cc2cc(O)cc(O)c2-4)-c2c(O)cc(O)cc2CC3c2ccc(O)cc2)cc1]

5. The composition of the agent for inducing differentiation of white adipocytes into brown-like adipocytes according to claim 4 wherein the phenol content in 20 mg/mL solid is 100 μg/mL or less.

6. A food, pharmaceutical agent, and quasi drug comprising the agent for inducing differentiation of white adipocytes into brown-like adipocytes according to claim 1 or 2.

7. A food, pharmaceutical agent, and quasi drug comprising the composition of the agent for inducing differentiation of white adipocytes into brown-like adipocytes according to claim 4 or 5.

8. A method for producing the composition of the agent for inducing differentiation of white adipocytes into brown-like adipocytes according to claim 4 or 5 which comprises a step for removing phenol contained therein by one or more steps selected from the group consisting of Steps (a) to (c):
(a) a step for allowing a solution obtained by heating resveratrol under an alkaline condition to be brought into contact with a solid adsorbent and recovering the adsorbed constituents;
(b) a step for adjusting a solution obtained by heating resveratrol under an alkaline condition to pH7 or below to effect precipitation and recovering the precipitated material; and,
(c) a step for heating a solution obtained by heating resveratrol under an alkaline condition, the composition obtained by the Step (a), or an aqueous solution containing the composition obtained by the Step (b).
